(19) United States
(12) Patent Application Publication
(10) Pub. No.: US 2013/0317055 A1
(43) Pub. Date: Nov. 28, 2013

(54) NEURONAL NICOTINIC AGONIST AND METHODS OF USE

(71) Applicant: ABBVIE INC., North Chicago, IL (US)

(72) Inventors: George M. HAIG, Gurnee, IL (US); Jeffrey D. BAKER, Evanston, IL (US)

(73) Assignee: ABBVIE INC., NORTH CHICAGO, IL (US)

(21) Appl. No.: 13/801,660

(22) Filed: Mar. 13, 2013

Related U.S. Application Data
(60) Provisional application No. 61/651,385, filed on May 24, 2012.
(60) Provisional application No. 61/681,382, filed on Aug. 9, 2012.

(51) Int. Cl. A61K 31/439

(52) U.S. Cl. CPC A61K 31/439
USPC 514/294; 546/94

(57) ABSTRACT

An embodiment relates to a selective agonist of neuronal nicotinic acetylcholine receptor α7 subtype, a pharmaceutically suitable salt, prodrug, or a metabolite thereof, for the prevention and treatment of diseases and conditions that are mediated by nicotinic acetylcholine receptors, and methods of use thereof. Another embodiment is a method of administering a pharmaceutically effective amount of a selective agonist of neuronal nicotinic acetylcholine receptor α7 subtype, a pharmaceutically suitable salt, prodrug, or a metabolite thereof, to a mammal in need thereof.

### CROSS-REFERENCE TO RELATED APPLICATIONS

This patent application claims the benefit of U.S. Provisional Application No. 61/651,385, filed on May 24, 2012, and U.S. Provisional Application No. 61/681,382, filed on Aug. 9, 2012, each of which is herein incorporated by reference in its entirety.

### STATEMENT REGARDING FEDERALLY SPONSORED RESEARCH OR DEVELOPMENT

Not applicable.

### FIELD OF THE INVENTION

The present disclosure neuronal nicotinic receptor agonists selective for a7 subtype that are useful for improving cognitive symptoms in patients having schizophrenia. Compounds, compositions containing such compounds, and methods of using such compound and compositions are described herein.

### BACKGROUND OF THE INVENTION

Nicotinic acetylcholine receptors (nAChRs) are widely distributed throughout the central (CNS) and peripheral (PNS) nervous systems. Such receptors play an important role in regulating CNS function, particularly by modulating release of a wide range of neurotransmitters, including, but not necessarily limited to, acetylcholine, norepinephrine, dopamine, serotonin, and GABA. Consequently, nicotinic receptors mediate a very wide range of physiological effects, and have been targeted for therapeutic treatment of disorders relating to cognitive function, learning and memory, neurodegeneration, pain, inflammation, psychosis, sensory gating, mood, and emotion, among other conditions.

Many subtypes of the nAChR exist in the CNS and periphery. Each subtype has a different effect on regulating the overall physiological function. Typically, nAChRs are ion channels that are constructed from a pentameric assembly of subunit proteins. At least 12 subunit proteins, α2-α10 and β2-β4, have been identified in neuronal tissue. These subunits provide for a great variety of homomeric and heteromeric combinations that account for the diverse receptor subtypes. For example, the predominant receptor that is responsible for high affinity binding of nicotine in brain tissue has composition (α4)2(β2)3 (the α4β2 subtype), while another major population of receptors is comprised of homomeric (α7)5 (the α7 subtype) receptors.

Certain compounds, like the plant alkaloid nicotine, interact with all subtypes of the nAChRs, accounting for the profound physiological effects of this compound. While nicotine has been demonstrated to have many beneficial properties, not all of the effects mediated by nicotine are desirable. For example, nicotine exerts gastrointestinal and cardiovascular side effects that interfere at therapeutic doses, and its addictive nature and acute toxicity are well-known. Ligands that select for interaction with only certain subtypes of the nAChR offer potential for achieving beneficial therapeutic effects with an improved margin for safety.

The α7 and α4β2 nAChRs have been shown to play a significant role in enhancing cognitive function, including aspects of learning, memory and attention (Levin, J. Neurobiol. 53: 633-640, 2002). For example, α7 nAChRs have been linked to conditions and disorders related to attention deficit disorder, attention deficit hyperactivity disorder (ADHD), schizophrenia, Alzheimer's disease (AD), mild cognitive impairment, senile dementia, dementia associated with Lewy bodies, dementia associated with Down's syndrome, AIDS dementia, and Pick's disease, as well as inflammation. The α4β2 receptor subtype is implicated in attention, cognition, epilepsy, and pain control (Paterson and Norberg, Progress in Neurobiology 61 75-111, 2000) as well as smoking cessation or nicotine withdrawal syndrome.

The activity at both α7 and α4β2 nAChRs can be modified or regulated by the administration of subtype selective nAChR ligands. The ligands can exhibit antagonist, agonist, or partial agonist properties. Compounds that function as allosteric modulators are also known.

Although compounds that nonselectively demonstrate activity at a range of nicotinic receptor subtypes including α7 nAChRs are known, it would be beneficial to provide compounds that interact selectively with α7-containing neuronal nAChRs compared to other subtypes.

It would be beneficial to provide such nicotinic acetylcholine receptor ligand for improving symptoms associated with nAChR-mediated conditions, for example disorders such as schizophrenia and other related disorders. There remains a need for providing a neuronal nicotinic acetylcholine receptor agonist that treats such conditions in a safe and efficacious manner.

### SUMMARY OF THE INVENTION

It has been found that α7 nicotinic acetylcholine receptor (nAChR) ligands, such as (4s)-4-(5-phenyl-1,3,4-thiadiazol-2-yloxy)-1-azatricyclo[3.3.1.1^{3,7}]decane, N-[2-(pyrdin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide or salts thereof are effective for improving symptoms of cognitive deficits associated with schizophrenia in human adult patients. Moreover, administration of (4s)-4-(5-phenyl-1,3,4-thiadiazol-2-yloxy)-1-azatricyclo[3.3.1.1³⁷]decane to human patients reduced the severity of symptoms associated with schizophrenia disease in patients in a generally well tolerated manner. (4s)-4-(5-Phenyl-1,3,4-thiadiazol-2-yloxy)-1-azatricyclo[3.3.1.1^{3,7}]decane (Compound A), a neuronal nicotinic receptor agonist selective for α7 subtype of nicotinic acetylcholine receptors demonstrated precognitive effects in patients with schizophrenia.

Other disorders that share common features with schizophrenia can be treated with administration of a neuronal nicotinic receptor agonist selective for α7 subtype. Schizophreniform disorder shares common symptoms with schizophrenia, however, the patient may demonstrate a shorter duration of disruptive symptoms and the patient's level of functioning may be less affected than a patient diagnosed with schizophrenia. Schizoaffective disorder has features of schizophrenia and an affective (or mood) disorder. Accordingly, schizophreniform, schizoaffective disorder and other disorders that belong to the schizophrenia spectrum of psychotic disorders can be treated with a neuronal nicotinic receptor agonist selective for α7 subtype, such as (4s)-4-(5-phenyl-1,3,4-thiadiazol-2-yloxy)-1-azatricyclo[3.3.1.1^{3,7}]decane, N-[2-(pyridin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide or salts thereof. Examples of additional disorders associated with the schizophrenia spectrum of psychotic disorders include, but are not limited to, schizotypal personality disorder, brief psychotic disorder, delusional disorder, and substance-induced psychotic disorder. Schizophrenia, schizophreniform, schizoaffective disorder, schizotypal personality disorder, brief psychotic disorder delusional disorder, and substance-induced psychotic disorder are collectively referred to as schizophrenia spectrum psychotic disorders.

A suitable medicament that is a neuronal nicotinic receptor agonist selective for α7 subtype is administered in sufficient doses to achieve therapeutic effect in a patient. (4s)-4-(5-Phenyl-1,3,4-thiadiazol-2-yloxy)-1-azatricyclo[3.3.1.1^{3,7}]decane can be administered to a patient in need of treatment in doses of from about 6 mg to about 150 rug once daily (QD). Examples of suitable doses in the range of doses that can be administered are 10 mg QD, 25 mg QD, 50 mg QD, and 75 mg QD. The medicament is administered in a suitable fashion to achieve therapeutic effect. Once daily dosing for (4s)-4-(5-phenyl-1,3,4-thiadiazol-2-yloxy)-1-azatricyclo[3.3.1.1^{3,7}]decane is achieved via oral administration.

The invention is a method of treating a patient in need of treatment for cognitive symptoms associated with schizophrenia, schizophreniform disorder, schizoaffective disorder, schizotypal personality disorder, brief psychotic disorder, delusional disorder, or substance-induced psychotic disorder. The patient is treated by administering a selective agonist of neuronal nicotinic acetylcholine receptor α7 subtype in a therapeutically effective amount. The patient can be treatment naïve, previously received treatment for schizophrenia or a related schizophrenia spectrum disorder, or is currently receiving treatment for schizophrenia or a related schizophrenia spectrum psychotic disorder. A suitable selective agonist of neuronal nicotinic acetylcholine receptor α7 subtype is (4s)-4-(5-phenyl-1,3,4-thiadiazol-2-yloxy)-1-azatricyclo[3.3.1.1^{3,7}]decane. The compound can be administered to the patient in doses of from about 6 mg to about 150 mg once daily, and more particularly at 10 mg QD, 25 rug QD, 50 rug QD, or 75 rug QD.

In one aspect, the invention relates to a method of improving symptoms of cognitive deficits associated with schizophrenia, schizophreniform disorder schizoaffective disorder, schizotypal personality disorder, brief psychotic disorder, delusional disorder, or substance-induced psychotic disorder, in a patient, comprising administering a selective agonist of neuronal nicotinic acetylcholine receptor α7 subtype to the patient.

In another aspect, the invention relates to a method of improving symptoms of cognitive deficits associated with schizophrenia, schizophreniform disorder, schizoaffective disorder, schizotypal personality disorder, brief psychotic disorder, delusional disorder, or substance-induced psychotic disorder, in a patient, comprising administering a selective agonist of neuronal nicotinic acetylcholine receptor α7 subtype to the patient, wherein the selective agonist of neuronal nicotinic acetylcholine receptor α7 subtype is (4s)-4-(5-phenyl-1,3,4-thiadiazol-2-yloxy)-1-azatricyclo[3.3.1.1^{3,7}]decane, N-[2-(pyridin-3-ylmethyl)-1-azabicyclo[2.2.21]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide or salts thereof.

Another aspect of the invention relates to use of a selective agonist of neuronal nicotinic acetylcholine receptor α7 subtype for preparation of a medicament for treating cognitive function associated with schizophrenia, schizophreniform disorder, schizoaffective disorder, schizotypal personality disorder, brief psychotic disorder, delusional disorder, or substance-induced psychotic disorder.

in another aspect, the invention relates to use of a selective agonist of neuronal nicotinic acetylcholine receptor α7 subtype for preparation of a medicament for treating cognitive function associated with schizophrenia, schizophreniform disorder, schizoaffective disorder, schizotypal personality disorder, brief psychotic disorder, delusional disorder, or substance-induced psychotic disorder, wherein the selective agonist of neuronal nicotinic acetylcholine receptor α7 subtype is (4s)-4-(5-phenyl-1,3,4-thiadiazol-2-yloxy)-1-azatricycio[3.1.1.1^{3,7}]decane, N-[2-(pyridin-3-ylmethyl)-1-azabicyclo[2.2.2]oct-3-yl]-1-benzofuran-2-carboxamide, N-[(3R)-1-azabicyclo[2.2.21]oct-3-yl]-7-chloro-1-benzothiophene-2-carboxamide, (R)-7-chloro-N-(quinuclidin-3-yl)benzo[b]thiophene-2-carboxamide or salts thereof.

Yet another aspect of the invention relates to pharmaceutical composition for use in the treatment of cognitive function associated with schizophrenia, schizophreniform disorder, schizoaffective disorder, schizotypal personality disorder, brief psychotic disorder, delusional disorder, or substance-induced psychotic disorder, comprising administering a therapeutically effective amount agonist of neuronal nicotinic acetylcholine receptor α7 subtype and a pharmaceutically acceptable excipient.

In yet another aspect, the invention relates to pharmaceutical composition for use in the treatment of cognitive function associated with schizophrenia, schizophreniform disorder, schizoaffective disorder, schizotypal personality disorder, brief psychotic disorder, delusional disorder, or substance-induced psychotic disorder, comprising administering a therapeutically effective amount agonist of neuronal nicotinic acetylcholine receptor α7 subtype and a pharmaceutically acceptable excipient, wherein the selective agonist of neuronal nicotinic acetylcholine receptor α7 subtype is (4s)-4-(5-phenyl-1,3,4-thiadiazol-2-yloxy)-1-azatricyclo[3.3.1.1^{3,7}]decane, N-[2-(pyridin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide or salts thereof.

Additional aspects of the invention and further details are provided herein.

### BRIEF DESCRIPTION OF THE DRAWINGS

FIG. 1 graphically depicts the mean change from baseline as measured by the MATRICS Consensus Cognitive Battery (MCCB) in patients administered (4S)-4-(5-phenyl-1,3,4-thiadiazol-2-yloxy)-1-azatricyclo[3.3.1.1^{3,7}]decane (Compound A) in stable subjects with schizophrenia receiving their antipsychotic treatment regimen in a double-blind, parallel-group Phase 2a clinical study when compared with placebo.

FIG. 2 graphically depicts the mean change from baseline in patients administered (4s)-4-(5-phenyl-1,3,4-thiadiazol-2-yloxy)-1-azatricyclo[3.3.1.1^{3,7}]decane (Compound A) in stable subjects with schizophrenia receiving their antipsychotic treatment regimen in a double-blind, parallel-group Phase 2a clinical study when compared with placebo as measured by the University of California at San Diego Performance-based Skills Assessment (version 2) (UPSA-2), a cognitive functional capacity measure. The UPSA-2 consists of 6 real-life specific cognitive tasks, each scored on a scale of 0-20 points. A composite score, which is the endpoint of interest, is derived by taking the sum of the scores from all 6 tasks.

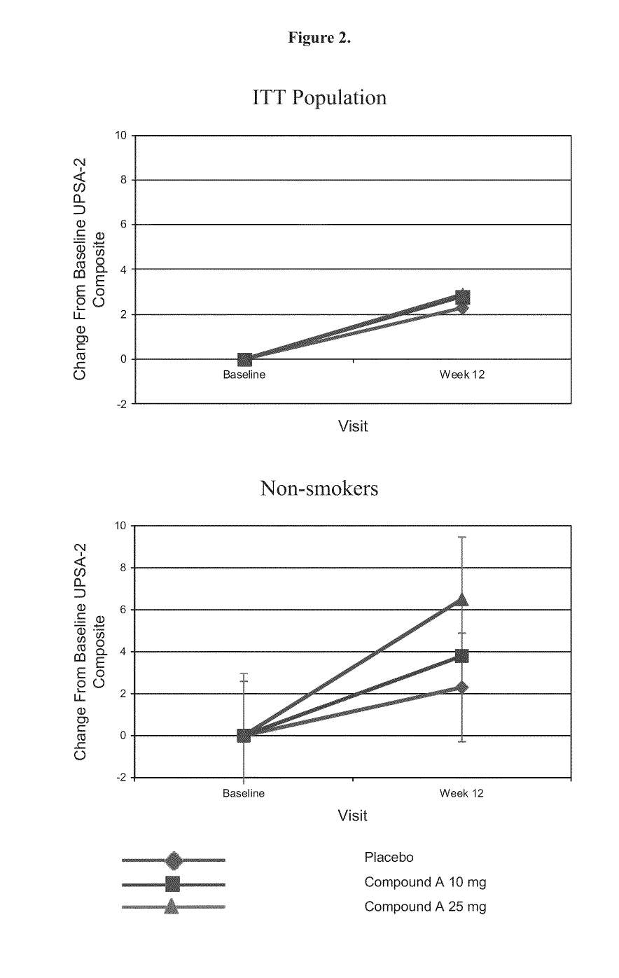

### DETAILED DESCRIPTION OF THE INVENTION

All patents, patent applications, and literature references cited in the specification are herein incorporated by reference in their entirety.

For a variable that occurs more than one time in any substituent or in the compound of the invention or any other formulae herein, its definition on each occurrence is independent of its definition at every other occurrence. Combinations of substituents are permissible only if such combinations result in stable compounds. Stable compounds are compounds which can be isolated in a useful degree of purity from a reaction mixture.

As used throughout this specification and the appended claims, the terms have the following meanings.

### Definition of Terms

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenylene” means a divalent group derived from a straight or branched chain hydrocarbon of from 2 to 10 carbon atoms containing at least one double bond. Representative examples of alkenylene include, but are not limited to, —CH═CH—, —CH═CH₂CH₂—, and —CH═C(CH₃)CH₂—.

The term “alkenyloxy” as used herein, means an alkenyl group, as defined herein, appended to the parent molecular moiety through an oxygen atom. Representative examples of alkenyloxy include, but are not limited to, allyloxy, 2-butenyloxy and 3-butenyloxy.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy” as used herein, means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkoxyalkyl” as used herein, means an alkoxyalkoxy group, as defined herein, appended to the parent molecular moiety through an alkyl group, as defined herein. Representative examples of alkoxyalkoxyalkyl include, but are not limited to, tert-butoxymethoxymethyl, ethoxymethoxymethyl, (2-methoxyethoxy)methyl, and 2-(2-methoxyethoxy)ethyl.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl” as used herein, means an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tort-butylcarbonyloxy.

The term “alkylene” means a divalent group derived from a straight or branched chain hydrocarbon of from 1 to 10 carbon atoms. Representative examples of alkylene include, but are not limited to, —CH₂—, —CH(CH₃)—, —C(CH₃)₂—, —CH₂CH₂—, —CH₂CH₂CH₂—, —CH₂CH₂CH₂CH₂—, and —CH₂CH(CH₃)CH₂—.

The term “alkylsulfinyl” as used herein, means an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfinylalkyl” as used herein, means an alkylsulfinyl group, as defined herein, appended to the parent molecular moiety through an alkyl group, as defined herein. Representative examples of alkylsulfinylalkyl include, but are not limited to, methylsulfinylmethyl and ethylsulfinylmethyl.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylsulfonylalkyl” as used herein, mean an alkylsulfonyl group, as defined herein, appended to the parent molecular moiety through an alkyl group, as defined herein. Representative examples of alkylsulfonylalkyl include, but are not limited to, methylsulfonylmethyl and ethylsulfonylmethyl.

The term “alkylthio” as used herein, means an alkyl group, as defined herein, appended to the parent molecular moiety through a sulfur atom. Representative examples of alkylthio include, but are not limited to, methylthio, ethylthio, tert-butylthio, and hexylthio.

The term “alkylthioalkyl” as used herein, means an alkylthio group, as defined herein, appended to the parent molecular moiety through an alkyl group, as defined herein. Representative examples of alkylthioalkyl include, but are not limited to, methylthiomethyl and 2-(ethylthio)ethyl.

The term “alkynyl” as used herein, means a straight or branched chain hydrocarbon group containing from 2 to 10 carbon atoms and containing at least one carbon-carbon triple bond. Representative examples of alkynyl include, but are not limited to, acetylenyl, 1-propynyl, 2-propynyl, 3-butynyl, 2-pentynyl, and 1-butynyl.

The term “alkynylene” means a divalent group derived from a straight or branched chain hydrocarbon of from 2 to 10 carbon atoms containing at least one triple bond. Representative examples of alkynylene include, but are not limited to, —C≡C—, —CH₂C≡C—, —-CH(CH₃)CH₂C≡C—, and —C≡CCH₂—, and —C≡CCH(CH₃CH₂—.

The term “alkynyloxy” as used herein, means an alkynyl group, as defined herein, appended to the parent molecular moiety through an oxygen atom. Representative examples of alkynyloxy include, but are not limited to, 2-propyhyloxy and 2-butynyloxy.

The term “aryl,” as used herein, means phenyl, a bicyclic and or a tricyclic aryl. The bicyclic aryl is naphthyl, a phenyl fused to a cycloalkyl, or a phenyl fused to a cycloalkenyl. Representative examples of the bicyclic aryl include, but are not limited to, dihydroindenyl, indenyl, naphthyl, dihydronaphthalenyl, and tetrahydronaphthalenyl. The tricyclic aryl is anthracene or phenanthrene, or a bicyclic aryl fused to a cycloalkyl, or a bicyclic aryl fused to a cycloalkenyl, or a bicyclic aryl fused to a phenyl. Representative examples of tricyclic aryl ring include, but are not limited to, azulenyl, dihydroanthracenyl, fluorenyl, and tetrahydrophenanthrenyl.

The aryl groups of this invention can be substituted with 1, 2, 3, 4 or 5 substituents independently selected from alkenyl, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, carboxy, carboxyalkyl, cyano, cyanoalkyl, formyl, formylalkyl, halogen, haloalkyl, hydroxy, hydroxyalkyl, mercapto, nitro, —NZ₁Z₂, and (NZ₃Z₄)carbonyl.

The term “arylalkoxy” as used herein, means an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “arylalkoxycarbonyl” as used herein, means an arylalkoxy group, as defined herein, appended to the parent molecular moiety through a carbonyl group, as defined herein, Representative examples of arylalkoxycarbonyl include, but are not limited to, benzyloxycarbonyl and naphth-2-ylmethoxycarbonyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylalkylthio” as used herein, means an arylalkyl group, as defined herein, appended to the parent molecular moiety through a sulfur atom. Representative examples of arylalkylthio include, but are not limited to, 2-phenylethylthio, 3-naphth-2-ylpropylthio, and 5-phenylpentylthio.

The term “arylcarbonyl” as used herein, means an aryl group, as defined herein, appended to the parent molecular moiety through a carbonyl group, as defined herein. Representative examples of arylcarbonyl include, but are not limited to, benzoyl and naphthoyl.

The term “aryloxy” as used herein, means an aryl group, as defined herein, appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “aryloxyalkyl” as used herein, means an aryloxy group, as defined herein, appended to the parent molecular moiety through an alkyl group, as defined herein. Representative examples of aryloxyalkyl include, but are not limited to, 2-phenoxyethyl, 3-naphth-2-yloxypropyl and 3-bromophenoxymethyl.

The term “arylthio” as used herein, means an aryl group, as defined herein, appended to the parent molecular moiety through a sulfur atom. Representative examples of arylthio include, but are not limited to, phenylthio and 2-naphthylthio.

The term “arylthioalkyl” as used herein, means an arylthio group, as defined herein, appended to the parent molecular moiety through an alkyl group, as defined herein. Representative examples of arylthioalkyl include, but are not limited to, phenylthiomethyl, 2-naphth-2-ylthioethyl, and 5-phenylthiomethyl.

The term “AUC_{∞}” refers to the area under the plasma concentration time curve (AUC) extrapolated to infinity.

The term “azido” as used herein, means a —N₃ group.

The term “carbonyl” as used herein, means a —C(O)— group.

The term “carboxy” as used herein, means a —CO₂H group.

The term “carboxyalkyl” as used herein, means a carboxy group, as defined herein, appended to the parent molecular moiety through an alkyl group, as defined herein, Representative examples of carboxyalkyl include, but are not limited to, carboxymethyl, 2-carboxyethyl, and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkenyl” as used herein, means a cyclic hydrocarbon containing from 3 to 8 carbons and containing at least one carbon-carbon double bond formed by the removal of two hydrogens. Representative examples of cycloalkenyl include, but are not limited to, 2-cyclohexen-1-yl, 3-cyclohexen-1-yl, 2,4-cyclohexadien-1-yl and 3-cyclopenten-1-yl.

The term “cycloalkyl” as used herein, means a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two adjacent or non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantane).

The cycloalkyl groups of the invention are optionally substituted with 1, 2, 3, 4 or 5 substituents selected from the group consisting of alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkylthioalkyl, alkynyl, carboxy, cyano, formyl, haloalkoxy, haloalkyl, halogen, hydroxy, hydroxyalkyl, mercapto, oxo, —NZ₁Z₂, and (NZ₃Z₄)carbonyl.

The term “cycloalkylalkyl” as used herein, means a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “cycloalkylcarbonyl” as used herein, means cycloalkyl group, as defined herein, appended to the parent molecular moiety through a carbonyl group, as defined herein, Representative examples of cycloalkylcarbonyl include, but are not limited to, cyclopropylcarbonyl, 2-cyclobutylcarbonyl, and cyclohexylcarbonyl.

The term “cycloalkyloxy” as used herein, means cycloalkyl group, as defined herein, appended to the parent molecular moiety through an oxygen atom, as defined herein. Representative examples of cycloalkyloxy include, but are not limited to, cyclopropyloxy, cyclobutyloxy, cyclopentyloxy, cyclohexyloxy, cycloheptyloxy, and cyclooctyloxy.

The term “cycloalkylthio” as used herein, means cycloalkyl group, as defined her in, appended to the parent molecular moiety through a sulfur atom, as defined herein, Representative examples of cycloalkylthio include, but are not limited to, cyclopropylthio, cyclobutylthio, cyclopentylthio, cyclohexylthio, cycloheptylthio, and cyclooctylthio.

The term “ethylenedioxy” as used herein, means —O(CH₂)₂O— group wherein the oxygen atoms of the ethylenedioxy group are attached to the parent molecular moiety through one carbon atom forming a 5 membered ring or the oxygen atoms of the ethylenedioxy group are attached to the parent molecular moiety through two adjacent carbon atoms forming a six membered ring.

The term “formyl” as used herein, means a —C(O)H group.

The term “formylalkyl” as used herein, means a formyl group, as defined herein, appended to the parent molecular moiety through an alkyl group, as defined herein. Representative examples of formylalkyl include, but are not limited to, formylmethyl and 2-formylethyl.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trilluoromethyl, pentafluoroethyl, and 2-chloro-3-fiuoropentyl.

The term “heteroaryl,” as used herein, means a monocyclic heteroaryl or a bicyclic heteroaryl. The monocyclic heteroaryl is a 5 or 6 membered ring that contains at least one heteroatom selected from the group consisting of nitrogen, oxygen and sulfur. The 5 membered ring contains two double bonds and the 6 membered ring contains three double bonds. The 5 or 6 membered heteroaryl is connected to the parent molecular moiety through any carbon atom or any substitutable nitrogen atom contained within the heteroaryl, provided that proper valance is maintained. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any substitutable nitrogen atom contained within the bicyclic heteroaryl, provided that proper valance is maintained. Representative examples of bicyclic heteroaryl include, but are not limited to, azaindolyl, benzimidazolyl, benzofuranyl, benzoxadiazolyl, benzoisoxazole, benzoisothiazole, benzooxazole, 1,3-benzothiazolyl, benzothienyl(or benzothiophenyl), cinnolinyl, furopyridine, indolyl, indazolyl, indolinonyl, isobenzofuran, isoindolyl, naphthyridinyl, oxadiazolyl, oxazolopyridine, quinolinyl, quinoxalinyl, thiadiazolyl, and thienopyridinyl.

The heteroaryl groups of the invention are optionally substituted with 1, 2, 3 or 4 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, nitro, —NZ₁Z₂ and (NZ₃Z₄)carbonyl. Heteroaryl groups of the invention that are substituted with a hydroxy group may be present as tautomers. The heteroaryl groups of the invention encompasse all tautomers including non-aromatic tautomers. In addition, the nitrogen heteroatoms can be optionally quaternized or oxidized to the N-oxide.

The term “heteroarylalkoxy” as used herein, means a heteroaryl group, as defined herein, appended to the parent molecular moiety through an alkoxy group, as defined herein. Representative examples of heteroarylalkoxy include, but are not limited to, fur-3-ylmethoxy, 1H-imidazol-2-ylmethoxy, 1H-imidazol-4-ylmethoxy, 1-(pyridin-4-yl)ethoxy, pyridin-3-ylmethoxy, 6-chloropyridin-3-ylmethoxy, pyridin-4-ylmethoxy, (6-(trifluoromethyl)pyridin-3-yl)methoxy, (6-(cyano)pyridin-3-yl)methoxy, (2-(cyano)pyridin-4-yl)methoxy, (5-(cyano)pyridin-2-yl)methoxy, (2-(chloro)pyridin-4-yl)methoxy, pyrimidin-5-ylmethoxy, 2-(pyrimidin-2-yl)propoxy, thien-2-ylmethoxy, and thien-3-ylmethoxy.

The term “heteroarylalkyl” as used herein, means a heteroaryl, as defined herein, appended to the parent molecular moiety through an alkyl group, as defined herein. Representative examples of heteroarylalkyl include, but are not limited to, fur-3-ylmethyl, 1H-imidazol-2-ylmethyl, 1H-imidazol-4-ylmethyl, 1-(pyridin-4-yl)ethyl, pyridin-3-ylmethyl, 6-chloropyridin-3-ylmethyl, pyridin-4-ylmethyl, (6-(trifluoromethyl)pyridin-3-yl)methyl, (6-(cyano)pyridin-3-yl)methyl, (2-(cyano)pyridin-4-yl)methyl, (5-(cyano)pyridin-2-yl)methyl, (2-(chloro)pyridin-4-yl)methyl, pyrimidin-5-ylmethyl, 2-(pyrimidin-2-yl)propyl, thien-2-ylmethyl, and thien-3-ylmethyl.

The term “heteroalalkylcarbonyl” as used herein, means a heteroarylalkyl, as defined herein, appended to the parent molecular moiety through a carbonyl group, as defined herein.

The term “heteroarylalkylthio” as used herein, means a heteroarylalkyl group, as defined herein, appended to the parent molecular moiety through a sulfur atom. Representative examples of heteroarylalkylthio include, but are not limited to, fur-3-ylmethylthio, 1H-imidazol-2-ylmethylthio, 1H-imidazol-4-ylmethylthio, pyridin-3-ylmethylthio, 6-chloropyridin-3-ylmethylthio, pyridin-4-ylmethylthio, (6-(trifluoromethyl)pyridin-3-yl)methylthio, (6-(cyano)pyridin-3-yl)methylthio, (2-(cyano)pyridin-4-yl)methylthio, (5-(cyano)pyridin-2-yl)methylthio, (2-(chloro)pyridin-4-ylmethylthio, pyrimidin-5-ylmethylthio, 2-(pyrimidin-2-yl)propylthio, thien-2-ylmethylthio, and thien-3-ylmethylthio.

The term “heteroarylcarbonyl” as used herein, means a heteroaryl group, as defined herein, appended to the parent molecular moiety through a carbonyl group, as defined herein. Representative examples of heteroarylcarbonyl include, but are not limited to, fur-3-ylcarbonyl, 1H-imidazol-2-ylcarbonyl, 1H-imidazol-4-ylcarbonyl, pyridin-3-ylcarbonyl, 6-chloropyridin-3-ylcarbonyl, pyridin-4-ylcarbonyl, (6-(trifluoromethyl)pyridin-3-yl)carbonyl, (6-(cyano)pyridin-3-yl)carbonyl, (2-(cyano)pyridin-4-yl)-carbonyl, (5-(cyano)pyridin-2-yl)carbonyl, (2-(chloro)pyridin-4-yl)carbonyl, pyrimidin-5-ylcarbonyl, pyrimidin-2-ylcarbonyl, thien-2-ylcarbonyl, and thien-3-ylcarbonyl.

The term “heteroaryloxy” as used herein, means a heteroaryl group, as defined herein, appended to the parent molecular moiety through an oxygen atom. Representative examples of heteroaryloxy include, but are not limited to, fur-3-yloxy, 1H-imidazol-2-yloxy, 1H-imidazol-4-yloxy, pyridin-3-yloxy, 6-chloropyridin-3-yloxy, pyridin-4-yloxy, (6-(trifluoromethyl)pyridin-3-yl)oxy, (6-(cyano)pyridin-3-yl)oxy, (2-(cyano)pyridin-4-yl)oxy, (5-(cyano)pyridin-2-yl)oxy, (2-(chloro)pyridin-4-yl)oxy pyrimidin-5-yloxy, pyrimidin-2-yloxy, thien-2-yloxy, and thien-3-yloxy.

The term “heteroaryloxyalkyl” as used herein, means a heteroaryloxy group, as defined herein, appended to the parent molecular moiety through an alkyl group, as defined herein. Representative examples of heteroaryloxyalkyl include, but are not limited to, pyridin-3-yloxymethyl and 2-quinolin-3-yloxyethyl.

The term “heteroarylthio” as used herein, means a heteroaryl group, as defined herein, appended to the parent molecular moiety through a sulfur atom. Representative examples of heteroarylthio include, but are not limited to, pyridin-3-ylthio and quinolin-3-ylthio.

The term “heteroarylthioalkyl” as used herein, means a heteroarylthio group, as defined herein, appended to the parent molecular moiety through an alkyl group, as defined herein. Representative examples of heteroarylthioalkyl include, but are not limited to, pyridin-3-ylthiomethyl, and 2-quinolin-3-ylthioethyl.

The term “heterocycle” or “heterocyclic” as used herein, means a monocyclic heterocycle, a bicyclic heterocycle or a tricyclic heterocycle. The monocyclic heterocycle is a 3, 4, 5, 6 or 7 membered ring containing at least one heteroatom independently selected from the group consisting of O, N, and S. The 3 or 4 membered ring contains 1 heteroatom selected from the group consisting of O, N and S. The 5 membered ring contains zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolidinyl, thiomorpholinyl, 1,1-dioxidothiomorpholinyl (thiomorpholine sulfone), thiopyranyl, and trithianyl. The bicyclic heterocycle is a 5 or 6 membered monocyclic heterocycle fused to a phenyl group, or a 5 or 6 membered monocyclic heterocycle fused to a cycloalkyl, or a 5 or 6 membered monocyclic heterocycle fused to a cycloalkenyl, or a 5 or 6 membered monocyclic heterocycle fused to a monocyclic heterocycle. The bicyclic heterocycle is connected to the parent molecular moiety through any carbon atom or any nitrogen atom contained within the bicyclic heterocycle. Representative examples of bicyclic heterocycle include, but are not limited to, 1,3-benzodioxolyl, 1,3-benzodithiolyl, 2,3-dihydro-1,4-benzodioxinyl, benzodioxolyl, 2,3-dihydro-1-benzofuranyl, 2,3-dihydro-1-benzothienyl, chromenyl and 1,2,3,4-tetrahydroquinolinyl. The tricyclic heterocycle is a bicyclic heterocycle fused to a phenyl, or a bicyclic heterocycle fused to a cycloalkyl, or a bicyclic heterocycle fused to a cycloalkenyl, or a bicyclic heterocycle fused to a monocyclic heterocycle. The tricyclic heterocycle is connected to the parent molecular moiety through any carbon atom or any nitrogen atom contained within the tricyclic heterocycle. Representative examples of tricyclic heterocycle include, but are not limited to, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b,d]thienyl.

The heterocycles of this invention are optionally substituted with 1, 2, 3 or 4 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, oxo, —NZ₁Z₂ and (NZ₃Z₄)carbonyl.

The term “heterocyclealkoxy” as used herein, means a heterocycle group, as defined herein, appended to the parent molecular moiety through an alkoxy group, as defined herein. Representative examples of heterocyclealkoxy include, but are not limited to, 2-pyridin-3-ylethoxy, 3-quinolin-3-ylpropoxy, and 5-pyridin-4-ylpentyloxy.

The term “heterocyclealkyl” as used herein, means a heterocycle, as defined herein, appended to the parent molecular moiety through an alkyl group, as defined herein, Representative examples of heterocyclealkyl include, but are not limited to, piperidin-4-ylmethyl, piperazin-1-ylmethyl, 3-methyl-1-pyrrolidin-1-ylbutyl, (1R)-3-methyl-1-pyrrolidin-1-ylbutyl, (1S)-3-methyl-1-pyrrolidin-1-ylbutyl.

The term “heterocyclealkylcarbonyl” as used herein, means a heterocyclealkyl, as defined herein, appended to the parent molecular moiety through a carbonyl group, as defined herein. Representative examples of heterocyclealkylcarbonyl include, but are not limited to, piperidin-4-ylmethylcarbonyl, piperazin-1-ylmethylcarbonyl, 3-methyl-1-pyrrolidin-1-ylbutylcarbonyl, (1R)-3-methyl-1-pyrrolidin-1-ylbutylcarbonyl, (1S)-3-methyl-1-pyrrolidin-1-ylbutylcarbonyl.

The term “heterocyclealkylthio” as used herein, means a heterocyclealkyl group, as defined herein, appended to the parent molecular moiety through a sulfur atom. Representative examples of heterocyclealkylthio include, but are not limited to, 2-pyridin-3-ylethylhio, 3-quinolin-3-ylpropythio, and 5-pyridin-4-ylpentylthio.

The term “heterocyclecarbonyl” as used herein, means a heterocycle, as defined herein, appended to the parent molecular moiety through a carbonyl group, as defined herein.

The term “heterocyclecarbonylalkyl” as used herein, means a heterocyclecarbonyl, as defined herein, appended to the parent molecular moiety through an alkyl group, as defined herein.

The term “heterocycleoxy” as used herein, means a heterocycle group, as defined herein, appended to the parent molecular moiety through an oxygen atom. Representative examples of heterocycleoxy include, but are not limited to, pyridin-3-yloxy and quinolin-3-yloxy.

The term “heterocycleoxyalkyl” as used herein, means a heterocycleoxy group, as defined herein, appended to the parent molecular moiety through an alkyl group, as defined herein. Representative examples of heterocycleoxyalkyl include, but are not limited to, pyridin-3-yloxymethyl and 2-quinolin-3-yloxyethyl.

The term “heterocyclethio” as used herein, means a heterocycle group, as defined herein, appended to the parent molecular moiety through a sulfur atom. Representative examples of heterocyclethio include, but are not limited to, pyridin-3-ylthio and quinolin-3-ylthio.

The term “heterocyclethioalkyl” as used herein, means a heterocyclethio group, as defined herein, appended to the parent molecular moiety through an alkyl group, as defined herein. Representative examples of heterocyclethioalkyl include, but are not limited to, pyridin-3-ylthiomethyl, and 2-quinolin-3-ylthioethyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “hydroxy-protecting group” or “O-protecting group” means a substituent which protects hydroxy groups against undesirable reactions during synthetic procedures. Examples of hydroxy-protecting groups include, but are not limited to, substituted methyl ethers, for example, methoxymethyl, benzyloxymethyl, 2-methoxyethoxymethyl, 2-(trimethylsilyl)-ethoxymethyl, benzyl, and triphenylmethyl; tetrahydropyranyl ethers; substituted ethyl ethers, for example, 2,2,2-trichloroethyl and t-butyl; silyl ethers, for example, trimethylsilyl, t-butyldimethylsilyl and t-butyldiphenylsilyl; cyclic acetals and ketals, for example, methylene acetal, acetonide and benzylidene acetal; cyclic ortho esters, for example, methoxymethylene; cyclic carbonates; and cyclic boronates. Commonly used hydroxy-protecting groups are disclosed in T. W. Greene and P. G. M. Wuts, Protective Groups in Organic Synthesis, 3rd edition, John Wiley & Sons, New York (1999).

The term “lower alkenyl” as used herein, is a subset of alkenyl, as defined herein, and means an alkenyl group containing from 2 to 4 carbon atoms. Examples of lower alkenyl are ethenyl, propenyl, and butenyl.

The term “lower alkoxy” as used herein, is a subset of alkoxy, as defined herein, and means a lower alkyl group, as defined herein, appended to the parent molecular moiety through an oxygen atom, as defined herein. Representative examples of lower alkoxy include, but are not limited to, methoxy, ethoxy, propoxy, 2-propoxy, butoxy, and tert-butoxy.

The term “lower alkyl” as used herein, is a subset of alkyl as defined herein and means a straight or branched chain hydrocarbon group containing from 1 to 4 carbon atoms. Examples of lower alkyl are methyl, ethyl, n-propyl, iso-propyl, n-butyl, iso-butyl, sec-butyl, and tert-butyl.

The term “lower alkylthio” as used herein, is a subset of alkylthio, means a lower alkyl group, as defined herein, appended to the parent molecular moiety through a sulfur atom. Representative examples of lower alkylthio include, but are not limited to, methylthio, ethylthio, and tert-butylthio.

The term “lower alkynyl” as used herein, is a subset of alkynyl, as defined herein, and means an alkynyl group containing from 2 to 4 carbon atoms. Examples of lower alkynyl are ethynyl, propynyl, and butynyl.

The term “lower haloalkoxy” as used herein, is a subset of haloalkoxy, as defined herein, and means a straight or branched chain haloalkoxy group containing from 1 to 4 carbon atoms. Representative examples of lower haloalkoxy include, but are not limited to, trifluoromethoxy, trichloromethoxy, dichloromethoxy, fluoromethoxy, and pentafluoroethoxy.

The term “lower haloalkyl” as used herein, is a subset of haloalkyl, as defined herein, and means a straight or branched chain haloalkyl group containing from 1 to 4 carbon atoms. Representative examples of lower haloalkyl include, but are not limited to, trifluoromethyl, trichloromethyl, dichloromethyl, fluoromethyl, and pentafluoroethyl.

The term “mercapto” as used herein, means a —SH group.

The term “mercaptoalkyl” as used herein, means a mercapto group, as defined herein, appended to the parent molecular moiety through an alkyl group, as defined herein. Representative examples of mercaptoalkyl include, but are not limited to, 2-mercaptoethyl and 3-mercaptopropyl.

The term “methylenedioxy” as used herein, means a —OCH₂O— group wherein the oxygen atoms of the methylenedioxy are attached to the parent molecular moiety through two adjacent carbon atoms.

The term “nitrogen protecting group” as used herein, means those groups intended to protect an amino group against undesirable reactions during synthetic procedures. Preferred nitrogen protecting groups are acetyl, benzoyl, benzyl, benzyloxycarbonyl (Cbz), formyl, phenylsulfonyl, tert-butoxycarbonyl (Boc), tert-butylacetyl, trifluoroacetyl, and triphenylmethyl (trityl).

The term “nitro” as used herein, means a —NO₂ group.

The term “NZ₁Z₂” as used herein, means two groups, Z₁ and Z₂, which are appended to the parent molecular moiety through a nitrogen atom. Z₁ and Z₂ are each independently selected from the group consisting of hydrogen, alkyl, alkylcarbonyl, alkoxycarbonyl, aryl, arylalkyl, formyl and (NZ₅Z₆)carbonyl. In certain instances within the invention, Z₁ and Z₂ taken together with the nitrogen atom to which they are attached form a heterocyclic ring. Representative examples of NZ₁Z₂ include, but are not limited to, amino, methylamino, acetylamino, acetylmethylamino, phenylamino, benzylamino, azetidinyl, pyrrolidinyl and piperidinyl.

The term “NZ₃Z₄” as used herein, means two groups, Z₃ and Z₄, which are appended to the parent molecular moiety through a nitrogen atom. Z₃ and Z₄ are each independently selected from the group consisting of hydrogen, alkyl, aryl and arylalkyl. Representative examples of NZ₃Z₄ include, but are not limited to, amino, methylamino, phenylamino and benzylamino.

The term “NZ₅Z₆” as used herein, means two groups, Z₅ and Z₆, which are appended to the parent molecular moiety through a nitrogen atom. Z₅ and Z₆ are each independently selected from the group consisting of hydrogen, alkyl, aryl and arylalkyl. Representative examples of NZ₅Z₆ include, but are not limited to, amino, methylamino, phenylamino and benzylamino.

The term “(NZ₃Z₄)carbonyl” as used herein, means a NZ₃Z₄ group, as defined herein, appended to the parent molecular moiety through a carbonyl group, as defined herein. Representative examples of (NZ₃Z₄)carbonyl include, but are not limited to, aminocarbonyl, (methylamino)carbonyl, (dimethylamino)carbonyl, and (ethylmethylamino)carbonyl.

The term “oxo” as used herein, means a ═O moiety.

The term “sulfinyl” as used herein, means a —S(O)— group.

The term “sulfonyl” as used herein, means a —SO₂— group.

The term “tautomer” as used herein means a proton shift from one atom of a compound to another atom of the same compound wherein two or more structurally distinct compounds are in equilibrium with each other.

The term “pharmaceutically suitable excipient” refers to a solid, semi-solid or liquid fillers, diluents, encapsulating material, formulation auxiliary suitable for administering to a subject. Examples of pharmaceutically suitable excipients include, but are not limited to, sugars, cellulose and derivatives thereof, oils, glycols, solutions, buffers, colorants, releasing agents, coating agents, sweetening agents, flavoring agents, perfuming agents, and the like. Such therapeutic compositions may be administered parenterally, intracisternally, orally, rectally, intraperitoneally or by other dosage forms known in the art.

The term “therapeutically suitable metabolite” refers to a pharmaceutically active compound formed by the in vivo biotransformation of compounds of formula (I-V).

The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds that are rapidly transformed in vivo to the compounds of formula (I-V) for example, by hydrolysis in blood.

The term “prodrug,” refers to compounds that contain, but are not limited to, substituents known as “therapeutically suitable esters.” The term “therapeutically suitable ester,” refers to alkoxycarbonyl groups appended to the parent molecule on an available carbon atom. More specifically, a “therapeutically suitable ester,” refers to alkoxycarbonyl groups appended to the parent molecule on one or more available aryl, cycloalkyl and/or heterocycle groups as defined herein. Compounds containing therapeutically suitable esters are an example, but are not intended to limit the scope of compounds considered to be prodrugs. Examples of prodrug ester groups include pivaloyloxymethyl, acetoxymethyl, phthalidyl, indanyl and methoxymethyl, as well as other such groups known in the art. Other examples of prodrug ester groups are found in T. Higuchi and V. Stella, Pro-drugs as Novel Delivery Systems, Vol, 14 of the A.C.S. Symposium Series, and in Edward B. Roche, ed., Bioreversible Carriers in Drug Design, American Pharmaceutical Association and Pergamon Press, 1987.

The terms “weight percent” or “percent by wei ht” or “% by weight” or “wt %” denote the weight of an individual component in a composition or mixture as a percentage of the weight of the composition or mixture.

Substituents attached to a cyclic moiety, for instance a cycloalkyl, aryl, or heterocycloalkyl moiety, can be represented as not bound to any particular atom, but rather as attached to bonds that perpendicularly intersect a side of the cyclic group. This notation is meant to indicate that the substituent can be bound to one of two or more atoms of the cyclic group.

Although typically it may be recognized that an asterisk is used to indicate that the exact subunit composition of a receptor is uncertain, for example α3b4* indicates a receptor that contains the α3 and b4 proteins in combination with other subunits, the term α7 as used herein is intended to include receptors wherein the exact subunit composition is both certain and uncertain. For example, as used herein α7 includes homomeric (α7)5 receptors and α7* receptors, which denote a nAChR containing at least one α7 subunit.

### Compounds of the Invention

Compounds which may be used in the rat hods and compositions of the invention are those of the Formula (I),

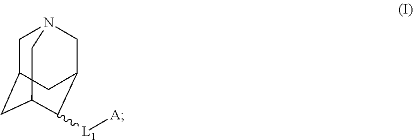

or a pharmaceutically acceptable salt or prodrug thereof, wherein

L₁ is —O— or —NRₐ—;

A is —Ar₁, —Ar₂-L₂-Ar₃ or —Ar₄-L₃-Ar₅;

Ar₁ is aryl or heteroaryl;

Ar₂ is aryl or monocyclic heteroaryl;

Ar₃ is aryl or heteroaryl;

Ar₄ is a bicyclic heteroaryl;

Ar₅ is aryl or heteroaryl;

L₂ is a bond, —O—, —NR—, —CH₂—, or —C(O)NRₐ—;

L₂ is a bond, —O—, —NR— or CH₂—; and

Rₐ is hydrogen or alkyl.

Another embodiment is a compound of formula (II),

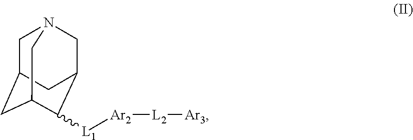

or a therapeutically suitable salt or prodrug thereof, wherein

Ar₂ is selected from

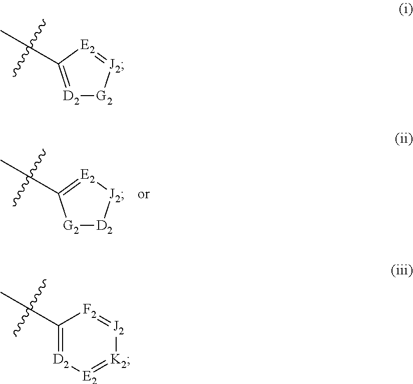

D₂, E₂, F₂, J₇, and K₂ are each independently —CT₂ or N;

G₂ is O, —NR₂ₐ, or S;

in each group of (i), (ii), and (iii), one substituent represented by T₂, or R₂ₐ wherein R₂ₐ is T₂, is -L₂-Ar₃ and the other substituents represented by T₂ are hydrogen, alkyl, alkoxy, alkoxycarbonyl, cyano, halo, nitro, or —NR_{b}R_{C};

R₂ₐ is hydrogen, alkyl, or T₂; and

R_{b} and R_{c} are each independently hydrogen, alkyl, alkoxycarbonyl or alkylcarbonyl.

Ar₃ is a group selected from

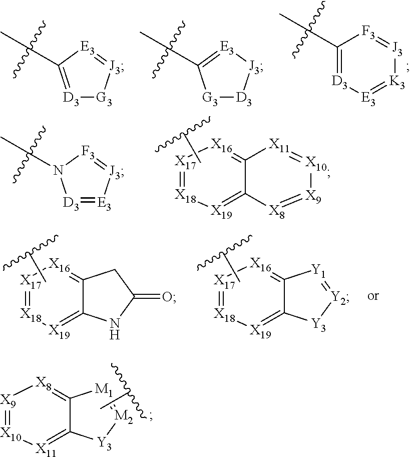

wherein D₃, E₃, F₃, J₃, K₃, X₈, X₉, X₁₀, and X₁₁ are each independently —CR₃ or N;

X₁₆, X₁₇, X₁₈, X₁₉, M₁, and M₂ are each independently —CR₃, N, or C;

G₃ is O, —NR₃ₐ, or S;

Y₁ is —CR₃ or N;

Y₂ is —CR₃ or N;

Y₃ is NH, O, or S;

R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano;

R₃ₐ is hydrogen, alkyl, alkylcarbonyl, tritylaryl, wherein aryl is preferably phenyl;

Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl;

one of X₁₆, X₁₇, X₁₈, and X₁₉, is C;

M₁ or M₂ is C;

L₁ is —O— or —NRₐ—;

L₂ is a bond, —O—, —NRₐ—, —CH₂—, or —C(O)NRₐ—; and

Rₐ is hydrogen or alkyl.

Another embodiment is a compound of formula (III),

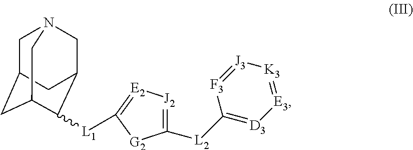

or a therapeutically suitable salt or prodrug thereof, wherein

E₂ and J₂ are each independently —CT₂ or N;

G₂ is O, —NR₂ₐ, or S;

T₂, at each occurrence, is independently hydrogen, alkyl, alkoxy, alkoxycarbonyl, cyano, halo, nitro, or —NR_{b}R_{c};

R₂ₐ is hydrogen, alkyl, or T₂;

R_{b} and R_{c} are each independently hydrogen, alkyl, alkoxycarbonyl or alkylcarbonyl;

D₃, E₃, F₃, J₃, and K₃ are each independently —CR₃ or N;

R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano;

Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl;

L₁ is —O— or —NRₐ—;

L₂ is a bond, —O—, —NRₐ—, —CH₂—, or —C(O)NRₐ—; and

Rₐ is hydrogen or alkyl.

Another embodiment is a compound of formula (IV),

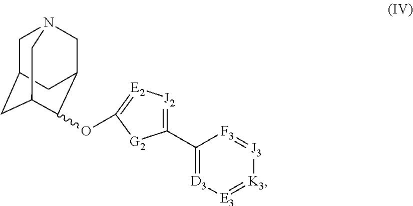

or a therapeutically suitable salt or prodrug thereof, wherein

E₂ and J₂ are each independently —CT₂ or N;

G₂ is —NR₂ₐ, or S;

T₂, at each occurrence, is independently hydrogen, alkyl, alkoxy, alkoxycarbonyl, cyano, halo, nitro, or NR_{b}R_{c};

R₂ₐ is hydrogen, alkyl, or T₂;

R_{b} and R_{c} are each independently hydrogen, alkyl, alkoxycarbonyl or alkylcarbonyl;

D₃, E₃, F₃, J₃, and K₃ are each independently —CR₃ or N;

R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano; and

Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl.

Another embodiment is a compound of formula (V),

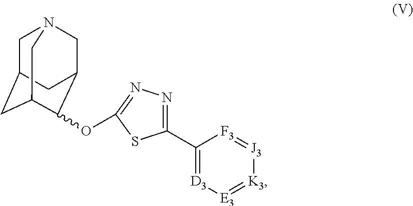

or a therapeutically suitable salt or prodrug thereof, wherein

D₃, E₃, F₃, J₃, and K₃ are each independently —CR₃ or N;

R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano; and

Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl.

Another embodiment is (4s)-4-(5-phenyl-1,3,4-thiadiazol-2-yloxy)-1-azatricyclo[3.3.1.1^{3,7}]decane (ABT-126 or Compound A).

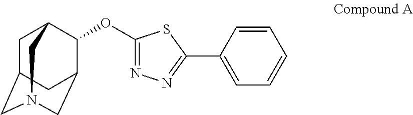 [SMILES: c1ccc(-c2nnc(O[C@@H]3C4CC5C[C@H]3C[N@](C5)C4)s2)cc1]

Alternatively, Compound A may also be called (1R,4R,5S)-4-(5-phenyl-1,3,4-thiadiazol-2-yloxy)-1-azatricyclo[3.3.1.1^{3,7}]decane or (E)-4-[(5-phenyl-1,3,4-thiadiazol-2-yl)oxy]-1-azoniatricyclo[3.3.1.1^{3,7}]decane 3,4-dicarboxy-3-hydroxybutanoate hydrate. The preparation of compounds of the invention is disclosed in US Patent Application Publication No. 20080167336.

The nAChR ligand agonist may be a compound of the Formula (VI),

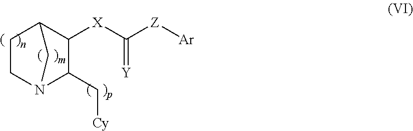

wherein in formula (VI)

m is 2;

n is 1;

p is 1, 2, 3 or 4;

X is oxygen or NR′;

Y is oxygen or sulfur;

Z is NR′, a covalent bond or a linker species A;

A is selected from the group —CR′R″—, —CR′R″—CR′R″—, —CR′═CR′— and —C═C—;

when Z is a covalent bond or A, X must be nitrogen;

Ar is an unsubstituted or substituted, carbocyclic or heterocyclic, monocyclic or fused polycyclic aryl group;

Cy is an unsubstituted or substituted 5- or 6-membered heteroaromatic ring; and substituents are selected from the group consisting of alkyl, alkenyl, heterocyclyl, cycloalkyl, aryl, substituted aryl, arylalkyl, substituted arylalkyl, halo, —OR′, —NR′R″, —CF3, —CN, —NO2, —R′, —SR′, —N3, —C(═O)NR′R″, —NR′C(═O)R″, —C(═O)R′, —C(═O)OR′, —OC(═O)R′, —O(CR′R″)rC(═O)R″, —O(CR′R″)rNR″C(═O)R′, —O(CR′R″)—NR″SO2R′, —OC(═O)NR′R″, —NR′C(═O)OR″, —SO2R′, —SO2NR′R″, and —NR′SO2R″;

wherein each of R′ and R″ individually is hydrogen, C₁-C₈ alkyl, C₃-C₈ cycloalkyl, heterocyclyl, aryl, or arylalkyl; or R′ and R″ can combine to form a 3 to 8 membered ring; and r is 1, 2, 3, 4, 5, or 6, or a pharmaceutically acceptable salt thereof.

Another compound which may be used for the methods may be TC-5619 (N-[2-(pyridin-3-ylmethyl)-1-azabicyclo[2.2.2]oct-3-yl]-1-benzofuran-2-carboxamide), which has been disclosed to be a neuronal nicotinic receptor agonist selective for α7 subtype.

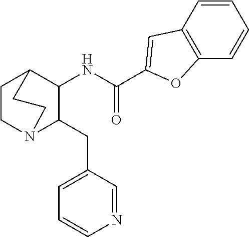 [SMILES: O=C(NC1C2CCN(CC2)C1Cc1cccnc1)c1cc2ccccc2o1]

The preparation of TC-5619 (N-[2-(pyridin-3-ylmethyl)-1-azabicyclo[2.2.2]oct-3-yl]-1-benzofuran-2-carboxamide) is disclosed U.S. Pat. No. 6,953,855.

The nAChR ligand agonist may be a compound of the Formula (VII),

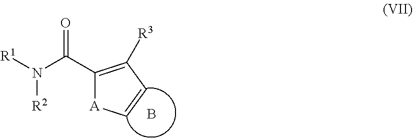

wherein in formula (VII)

R¹ represents 1-azabicyclo[2,2,2]oct-3-yl,

R² represents hydrogen or C₁-C₆-alkyl,

R³ represents hydrogen, halohalogen or C₁-C₆-alkyl,

A represents oxygen or sulfur, and

the ring B represents benzo, pyrimido, pyrimidazo or pyridazino which is substituted by a radical selected from the group consisting of halogen, C₁-C₆-alkanoyl, carbamoyl, cyano, trifluoromethyl, trifluoromethoxy, nitro, amino, C₁-C₆-acylamino, C₁-C₆-alkyoxy, C₁-C₆-alkylthio, C₁-C₆-alkylamino, heteroarylcarbonylamino, arylcarbonylamino, C₁-C₆-alkylsulfonyl-amino, di(C₁-C₄-alkylsulfonyl)amino, arylsulfonylamino, di(arylsulfonyl)amino, C₃-C₆-cycloalkylcarbonylmethyl, 1,3-dioxa-propane-1,3-diyl, amino(hydroxyimino)methyl and benzo, or a salt, a hydrate or a hydrate of a salt thereof.

The nAChR ligand agonist may be a compound of the Formula (VIII),

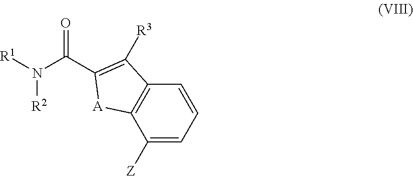

wherein in formula (VIII)

R¹ represents 1-azabicyclo[2,2,2]oct-3-yl,

R² represents hydrogen or C₁-C₆-alkyl,

R³ represents hydrogen, halogen or C₁-C₆-alkyl,

A represents oxygen or sulfur,

and Z represents halogen, formyl, carbamoyl, cyano, trifluoromethyl, trifluoromethoxy, nitro, amino, formamido, acetamido, C₁-C₆alkyl, C₁-C₆-alkyoxy, C₁-C₆-alkylthio, C₁-C₆-alkylamino, heteroaryl-carbonylamino, arylcarbonylamino, C₁-C₄-alkylsulfonylamino, di(arylsulfonyl)amino, C₃-C₆-cycloalkylcarbonylmethyl or amino(hydroxyimino)methyl, or a salt, a hydrate or a hydrate of a salt thereof.

Another compound which may be used for the methods may be EVP-6124, which has been disclosed to be a neuronal nicotinic receptor partial agonist selective for α7 subtype. The preparation of EVP-6124 (N-[(3R)-1-azabicyclo[2.2.2]oct-3-yl]-7-chloro-1-benzothiophene-2-carboxamide) is disclosed in U.S. Pat. No. 7,732,477.

The nAChR ligand agonist may be (R)-7-chloro-N-(quinuclidin-3-yl)benzo[b]thiophene-2-carboxamide and has the following structure:

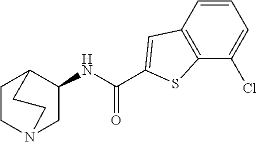 [SMILES: O=C(N[C@H]1CN2CCC1CC2)c1cc2cccc(Cl)c2s1]

### Salts of the Invention

The present compounds may exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts may be prepared during the final isolation and purification of the compounds or separately by reacting an amino group of the compounds with a suitable acid. For example, a compound may be dissolved in a suitable solvent, such as but not limited to methanol and water, and treated with at least one equivalent of an acid, like hydrochloric acid. The resulting salt may precipitate out and be isolated by filtration and dried under reduced pressure. Alternatively, the solvent and excess acid may be removed under reduced pressure to provide the salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, form ate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. The amino groups of the compounds may also be quaternized with alkyl chlorides, bromides, and iodides such as methyl, ethyl, propyl, isopropyl, butyl, lauryl, myristyl, stearyl, and the like.

Substantially pure crystalline salts of (4s)-4-(5-phenyl-1,3,4-thiadiazol-2-yloxy)-1-azatricyclo[3.3.1.1^{3,7}]decane are, for example, (4s)-4-(5-phenyl-1,3,4-thiadiazol-2-yloxy)-1-azatricyclo[3.3.1.1^{3,7}]decane L-bitartrate anhydrate, (4s)-4-(5-phenyl-1,3,4-thiadiazol-2-yloxy)-1-azatricyclo[3.3.1.1^{3,7}]decane L-bitartrate hydrate, (4s)-4-(5-phenyl-1,3,4-thiadiazol-2-yloxy)-1-azatricyclo[3.3.1.1^{3,7}]decane dihydrogen phosphate anhydrate, (4S)-4-(5-phenyl-1,3,4-thiadiazol-2-yloxy)-1-azatricyclo[3.3.1.1^{3,7}]decane dihydrogen phosphate hydrate, (4s)-4-(5-phenyl-1,3,4-thiadiazol-2-yloxy)-1-azatricyclo[3.3.1.1^{3,7}]decane bisuccinate anhydrate, (4s)-4-(5-phenyl-1,3,4-thiadiazol-2-yloxy)-1-azatricyclo[3.3.1.1^{3,7}]decane bisuccinate hydrate, (4s)-4-(5-phenyl-1,3,4-thiadiazol-2-yloxy)-1-azatricyclo[3.3.1.1^{3,7}]decane hydrochloride quartethydrate, (4s)-4-(5-phenyl-1,3,4-thiadiazol-2-yloxy)-1-azatricyclo[3.3.1.1^{3,7}]decane hydrochloride sesquihydrate, (4s)-4-(5-phenyl-1,3,4-thiadiazol-2-yloxy)-1-azatricyclo[3.3.1.1^{3,7}]decane monohydrogen citrate, (4s)-4-(5-phenyl-1,3,4-thiadiazol-2-yloxy)-1-azatricyclo[3.3.1.1^{3,7}]decane monohydrogen citrate, or (4s)-4-(5-phenyl-1,3,4-thiadiazol-2-yloxy)-1-azatricyclo[3.3.1.1^{3,7}]decane.

Basic addition salts may be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like, are contemplated as being within the scope of the present invention.

### Amides, Esters and Prodrugs of the Invention

Prodrugs are derivatives of an active drug designed to ameliorate some identified, undesirable physical or biological property. The physical properties are usually solubility (too much or not enough lipid or aqueous solubility) or stability related, while problematic biological properties include too rapid metabolism or poor bioavailability which itself may be related to a physicochemical property.

Prodrugs are usually prepared by: a) formation of ester, hemi esters, carbonate esters, nitrate esters, amides, hydroxamic acids, carbamates, imines, Mannich bases, and enamines of the active drug, b) functionalizing the drug with azo, glycoside, peptide, and ether functional groups, c) use of polymers, salts, complexes, phosphoramides, acetals, hemiacetals, and ketal forms of the drug. For example, see Andrejus Korolkovas's, “Essentials of Medicinal Chemistry”, John Wiley-Interscience Publications, John Wiley and Sons, New York (1988), pp. 97-118, which is incorporated in its entirety by reference herein.

Esters can be prepared from substrates of formula (I) containing either a hydroxyl group or a carboxy group by general methods known to persons skilled in the art. The typical reactions of these compounds are substitutions replacing one of the heteroatoms by another atom, for example:

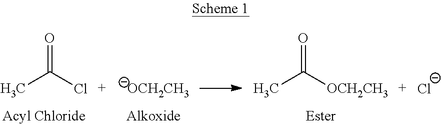

Amides can be prepared from substrates of formula (I) containing either an amino group or a carboxy group in similar fashion. Esters can also react with amines or ammonia to form amides.

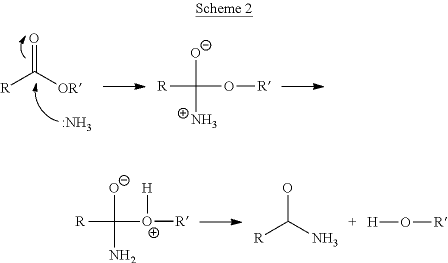

Another way to make amides from compounds of formula (I) to heat carboxylic acids and amines together.

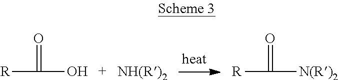

In Schemes 2 and 3, R and R′ are independently substrates of formulas I-V, alkyl or hydrogen.

### Optical Isomers-Diastereomers-Geometric Isomers

Asymmetric centers may exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described hereinbelow and resolved by techniques well known in the art.

Geometric isomers may exist in the present compounds. The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration. Furthermore, the invention contemplates the various isomers and mixtures thereof resulting from the disposal of substituents around an adamantane ring system. Two substituents around a single ring within an adamantine ring system are designated as being of Z or E relative configuration. For examples, see C. D. Jones, M. Kaselj, R. N. Salvatore, W. J. le Noble J. Org. Chem. 63: 2758-2760, 1998.

Compounds of the invention may exist as stereoisomers wherein, asymmetric or chiral centers are present. These stereoisomers are “R” or “S” depending on the configuration of substituents around the chiral element. The terms “R” and “S” used herein are configurations as defined in IUPAC 1974 Recommendations for Section E, Fundamental Stereochemistry, Pure Appl. Chem., 1976.45: 13-30. The invention contemplates various stereoisomers and mixtures thereof and are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss. Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

More particularly, the compounds of the invention can exist in the forms represented by formula (Ia) and (Ib).

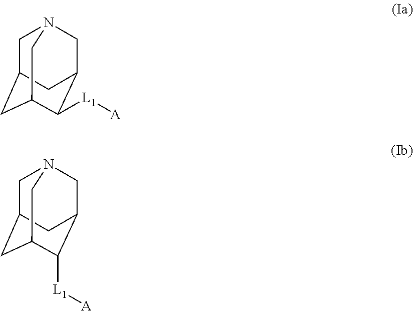

The aza-adamantane portion of isomer (Ia) and isomer (Ib) is not chiral, however the C-4 carbon at which L₁ is attached is considered pseudoasymmetric. Compounds represented by formula (Ia) and (Ib) are diastereomers. The configurational assignment of structures of formula (Ia) are assigned 4r in accordance with that described in Synthesis, 1992, 1080, Becker, D. P.; Flynn, D. L. and as defined in Stereochemistry of Organic Compounds, E. L. Eliel, S. H. Wilen; John Wiley and Sons, Inc. 1994. In addition the configurational assignment of structures of formula (Ib) are assigned 4s using the same methods.

The isomers (Ia) and (Ib) may be synthesized separately using the individual steroisomers according to the Schemes or the Experimentals described herein. Alternatively, isomers (Ia) and (Ib) may be synthesized together after which the individual isomers may be separated by chromatographic methods from the mixture of both isomers when mixtures of stereoisomers are used in the synthesis. The mixtures of isomers may also be separated through fractional crystallization of salts of amines contained in the compounds of formula (I) made with enantiomerically pure carboxylic acids.

It is contemplated that a mixture of both isomers may be used to modulate the effects of nAChRs. Furthermore, it is contemplated that the individual isomers of formula (Ia) and (Ib) may be used alone to modulate the effects of nAChRs. Therefore, it is contemplated that either a mixture of the compounds of formula (Ia) and (Ib) or the individual isomers alone represented by the compounds of formula (Ia) or (Ib) would be effective in modulating the effects of nAChRs, and more particularly α7 nAChRs, α4β2 nAChRs, or a combination of α7 nAChRs and α4β2 nAChRs and is thus within the scope of the invention.

More specifically, compounds contemplated as part of the invention include

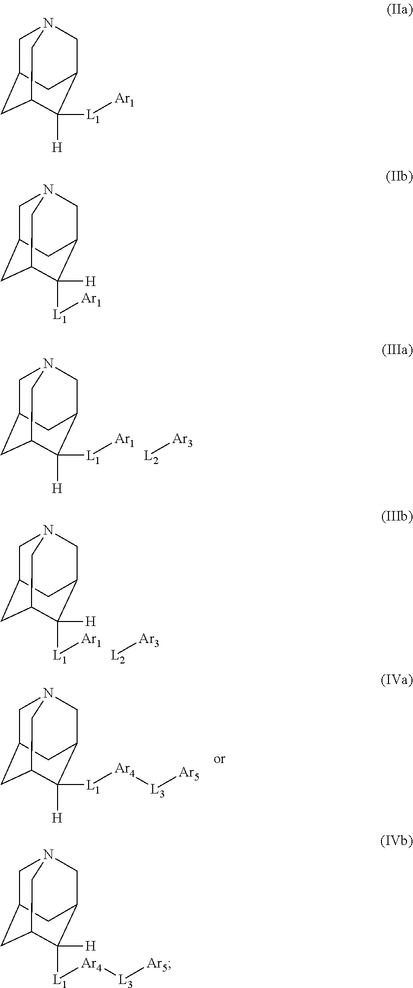

wherein L₁, L₂, L₃, Ar₁, Ar₂, Ar₃, Ar₄, and Ar₅ are defined herein.

### Isotope Enriched or Labeled Compounds

Compounds of the invention can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to, ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

In another embodiment, the isotope-labeled compounds contain deuterium (²H), tritium (³H) or ¹⁴C isotopes. Isotope-labeled compounds of this invention can be prepared by the general methods well known to persons having ordinary skill in the aft. Such isotope-labeled compounds can be conveniently prepared by carrying out the procedures disclosed in the Examples disclosed herein and Schemes by substituting a readily available isotope-labeled reagent for a non-labeled reagent. In some instances, compounds may be treated with isotope-labeled reagents to exchange a normal atom with its isotope, for example, hydrogen for deuterium can be exchanged by the action of a deuteric acid such as D₂SO₄/D₇O. In addition to the above, relevant procedures and intermediates are disclosed, for instance, in Lizondo, J et al., Drugs Fut, 21(11), 1116 (1996); Brickner, S J et al., J Med Chem, 39(3), 673 (1996); Mallesham, B et al., Org Lett, 5(7), 963 (2003); PCT publications WO1997010223, WO2005099353, WO1995007271, WO2006008754; U.S. Pat. Nos. 7,538,189; 7,534,814; 7,531,685; 7,528,131; 7,521,421; 7,514,068; 7,511,013; and US Patent Application Publication Nos. 20090137457; 20090131485; 20090131363; 20090118238; 20090111840; 20090105338; 20090105307; 20090105147; 20090093422; 20090088416; and 20090082471, the methods are hereby incorporated by reference.

The isotope-labeled compounds of the invention may be used as standards to determine the effectiveness of nAChR ligands in binding assays. Isotope containing compounds have been used in pharmaceutical research to investigate the in vivo metabolic fate of the compounds by evaluation of the mechanism of action and metabolic pathway of the nonisotope-labeled parent compound (Blake et al, J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al., J. Labelled Comp. Radiopharmaceut., 36(10):927-932 (1995); Kushner et al., Can. J. Physiol. Pharmacol., 77, 79-88 (1999).

In addition, non-radio active isotope containing drugs, such as deuterated drugs called “heavy drugs,” can be used for the treatment of diseases and conditions related to nAChR activity. Increasing the amount of an isotope present in a compound above its natural abundance is called enrichment. Examples of the amount of enrichment include from about 0, 5, 1, 2, 3, 4, 5, 6, 7, 8, 9, 10, 12, 16, 21, 25, 29, 33, 37, 42, 46, 50, 54, 58, 63, 67, 75, 79, 84, 88, 92, 96, to about 100 mol %. Replacement of up to about 15% of normal atom with a heavy isotope has been effected and maintained for a period of days to weeks in mammals, including rodents and dogs, with minimal observed adverse effects (Czajka D M and Finkel A J, Ann. N.Y. Acad. Sci. 1960 84: 770; Thomson J F, Ann. New York Acad. Sci. 1960 84: 736; Czakja D M et al., Am. J. Physiol. 1961 201: 357). Acute replacement of as high as 15%-23% in human fluids with deuterium was found not to cause toxicity (Blagojevic N et al. in “Dosimetry & Treatment Planning for Neutron Capture Therapy”, Zamenhof R, Solares G and Marling O Eds. 1994. Advanced Medical Publishing, Madison Wis. pp. 125-134; Diabetes Metab. 23: 251 (1997)).

Stable isotope labeling of a drug can alter its physico-chemical properties such as pKa and lipid solubility. These effects and alterations can affect the pharmacodynamic response of the drug molecule if the isotopic substitution affects a region involved in a ligand-receptor interaction. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. Accordingly, the incorporation of an isotope at a site of metabolism or enzymatic transformation will slow said reactions potentially altering the pharmacokinetic profile or efficacy relative to the non-isotopic compound.

### Compositions of the Invention

Therapeutic compositions of the disclosure comprise an effective amount of an nAChR ligands of formulas I-V, or pharmaceutically acceptable salts, prodrugs, esters, amides or metabolites thereof formulated with one or more therapeutically suitable excipients.

In one embodiment, the therapeutically effective amount comprises an amount of the nAChR ligand from about 6 mg to about 150 mg. In another embodiment the therapeutically effective amount is selected from the group consisting of about 10 mg to about 150 mg, 10 mg to about 75 mg, about 10 mg to about 50 mg, about 10 rug to about 25 mg, about 25 mg to about 150 mg, about 25 nag to about 75 nag, about 25 mg to about 50 mg, about 25 mg to about 50 mg, or about 50 mg to about 75 mg.

In another embodiment, the therapeutically effective amount of Compound A comprises an amount of the nAChR ligand from about 10 mg to about 150 mg. In another embodiment the therapeutically effective amount is selected from the group consisting of about 10 mg to about 150 nag, 10 mg to about 75 mg, about 10 mg to about 50 mg, about 10 mg to about 25 mg, about 25 mg to about 150 mg, about 25 rug to about 75 rug, about 25 rug to about 50 mg, about 25 nag to about 50 nag, or about 50 mg to about 75 mg.

In another embodiment, the therapeutically effective amount of Compound A comprises an amount of the nAChR ligand from about 25 mg to about 75 mg. Compound A is administered in doses of 10 mg, 25 mg, 50 mg, or 75 mg.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions can also contain adjuvants such as preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. It also can be desirable to include isotonic agents, for example, sugars, sodium chloride and the like. Prolonged absorption of the injectable pharmaceutical form can be brought about by the use of agents delaying absorption, for example, aluminum monostearate and gelatin.

In some cases, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug can depend upon its rate of dissolution, which, in turn, may depend upon crystal size and crystalline form. Alternatively, a parenterally administered drug form can be administered by dissolving or suspending the drug in an oil vehicle.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions can be formulated according to the known art using suitable dispersing or wetting agents and suspending agents. The sterile injectable preparation also can be a sterile injectable solution, suspension or emulsion in a nontoxic, parenterally acceptable diluent or solvent such as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, eardrops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can con ain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention also can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together.

Methods to form liposomes are known in the art. Sec, for example, Prescott, Ed., Methods in Cell Biology, Volume XIV, Academic Press, New York, N.Y., (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also contemplated as being within the scope of this invention. Aqueous liquid compositions of the invention also are particularly useful.

The compounds of the invention can be used in the form of pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

Representative acid addition salts can be prepared using various suitable acids for example, including, but are not limited to, acetic, adipic, alginic, citric, aspartic, benzoic, benzenesulfonic, butyric, camphoric, camphorsulfonic, carbonic, digluconic, glycerophosphoric, heptanoic, hexanoic, fumaric, hydrochloric, hydrobromic, hydroiodic, 2-hydroxyethansulfonic (isethionic), lactic, maleic, methanesulfonic, nicotinic, 2-naphthalenesulfonic, oxalic, pamoic, pectinic, persulfuric, 3-phenylpropionic, picric, pivalic, propionic, succinic, sulfuric, tartaric, thiocyanic, phosphoric, glutamatic, p-toluenesulfonic, and undecanoic acids.

Particular examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid, tartaric acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, myristyl and stearyl chlorides, bromides and iodides; arylalkyl halides such as benzyl and phenethyl bromides and others. Water or oil-soluble or dispersible products are thereby obtained.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention also contemplates pharmaceutically acceptable compounds that when administered to a patient in need may be converted through in vivo biotransformation into compounds of formula (I).

### METHODS OF THE PRESENT INVENTION

The compounds or compositions are administered to a patient in need of schizophrenia therapy or antipsychotic treatment. Such patient generally has received a diagnosis of schizophrenia. Any therapeutically effective neuronal nicotinic acetylcholine receptor agonist selective for α7 subtype can be administered to patients who are clinically stable and receiving a current regimen of atypical antipsychotic medications. Use in patients who have not yet received atypical antipsychotic medication or patients no longer receiving atypical antipsychotic medication also is contemplated.

The compound or composition is administered to a patient in need of treatment for schizophrenia or a disorder that is identified on the schizophrenia spectrum of psychotic disorders. Examples of disorders associated with the schizophrenia spectrum of psychotic disorders include, but are not limited to, schizotypal personality disorder, brief psychotic disorder, delusional disorder, and substance-induced psychotic disorder. Schizophrenia, schizophreniform, schizoaffective disorder, schizotypal personality disorder, brief psychotic disorder delusional disorder, and substance-induced pscythoic disorder are collectively referred to as schizophrenia spectrum psychotic disorders.

Patients are administered a therapeutically effective amount of a suitable compound or composition. In one embodiment, the therapeutically effective amount comprises an amount of the nAChR ligand from about 6 mg to about 150 mg. In another embodiment the therapeutically effective amount is selected from the group consisting of about 10 mg to about 150 mg, 10 mg to about 75 mg, about 10 mg to about 50 mg, about 10 mg to about 25 mg, about 25 mg to about 150 mg, about 25 mg to about 75 mg, about 25 mg to about 50 mg, about 25 mg to about 50 mg, or about 50 mg to about 75 mg.

In another embodiment, the therapeutically effective amount of Compound comprises an amount of the nAChR ligand from about 10 mg to about 150 mg. In another embodiment the therapeutically effective amount is selected from the group consisting of about 10 mg to about 150 mg, 10 mg to about 75 mg, about 10 mg to about 50 mg, about 10 mg to about 25 mg, about 25 mg to about 150 mg, about 25 mg to about 75 mg, about 25 rug to about 50 mg, about 25 mg to about 50 mg, or about 50 mg to about 75 mg.

In another embodiment, the therapeutically effective amount of Compound A comprises an amount of the nAChR ligand from about 25 mg to about 75 mg. Compound A is administered in doses of 10 mg, 25 mg, 50 mg, or 75 mg.

The compound or composition is delivered in a manner suitable for allowing the nAChR ligand to achieve therapeutic effect by interacting with the target receptor. The amount of the active agent administered can vary with the patient, the route of administration, and the result sought. Optimum dosing regimens for particular patients can be determined by one skilled in the art using the guidance and dosing information provided herein.

In accordance with the present invention, the active agent can be administered in any convenient manner. Examples of suitable methods for administration include, but are not limited, orally, sublingually, rectally, parentally, (including subcutaneously intrathechally, intramuscularly, and intravenously), or transdermally. The most preferred route of administration is the oral route.

The active agents of the invention can be administered in the form of pharmaceutical composition or compositions that contain one or both active agents in an admixture with a pharmaceutical carrier. The pharmaceutical composition can be in dosage unit form such as tablet, capsule, sprinkle capsule, granule, powder, syrup, or the like.

Certain aspects of the invention are described in greater detail in the non-limiting Examples that follow:

### Example 1

### Clinical Study A: Experimental Details

### Subjects

A Phase 2a proof-of-concept (POC) study in clinically stable subjects with schizophrenia who were clinically stable and received stable doses of atypical antipsychotic therapy. The study was a Phase 2, multi-center, randomized, double-blind, placebo-controlled, parallel group study designed to evaluate the safety and efficacy of doses of (4s)-4-(5-phenyl-1,3,4-thiadiazol-2-yloxy)-1-azatricyclo[3.3.1.1^{3,7}]decane (Compound A) in clinically stable male and female subjects (ages 20 to 55, inclusive) with a Diagnostic and Statistical Manual of Mental Disorders Fourth Edition, Text Revision (DSM-IV-TR) diagnosis of schizophrenia. Psychiatric diagnoses were confirmed using the Mini-International Neuropsychiatric Interview (MINI) version 6.0.0. The criteria for clinical stability was determined by a combination of retrospective data (over the 4 months prior to the start of Screening, which will be supported by clinical records, patient, identified responsible contact person, and physician interviews), and prospective data assessed during the Prospective Stabilization Period of 28 to 42 days duration.

### Study Design

The study was a Phase 2, multi-center, randomized, double-blind, placebo-controlled, parallel group study designed to evaluate the safety and efficacy of doses of (4s)-4-(5-phenyl-1,3,4-thiadiazol-2-yloxy)-1-azatrieyclo[3.3.1.1^{3,7}]decane, which is also recognized as Compound A, in treating cognitive deficits in subjects with schizophrenia who were clinically stable and receiving one or two atypical antipsychotic medications. Study drug was administered orally.

The study consisted of a screening period of at least 28 and up to 42 days, a 84-day outpatient treatment period, a 14-day post-treatment period, and a post-treatment follow-up period. The screening period consisted of three visits: Screening Visit 1, Screening Visit 2, and Day −1. Upon completion of Day −1 procedures, eligible subjects were randomized through an Interactive Voice Response/Interactive Web-Based (IVR/IWB) system. Subjects were randomized in an equal ratio to one of three treatment groups (placebo, 10 mg Compound A, or 25 mg Compound A).

### Inclusion Criteria for Study Subjects

Study subjects eligible for participation in the study met the following criteria during the screening period:
1. Male or female between 20 and 55 years of age, inclusive, at the time of randomization (Day −1).
2. Have a current DSM-IV-TR diagnosis of schizophrenia confirmed by the M.I.N.I. version 6.0.0.
3. Receiving an antipsychotic regimen of one or two atypical antipsychotic medications.
4. Is clinically stable in the residual phase of illness, as defined by the following criteria:
   Level of Care: The subject had no psychiatric inpatient hospitalization, no overnight crisis stabilization, no emergency room visit for psychiatric symptoms, and no other overt signs of destabilization from 4 months prior to the Initial Screening Visit.
   Stability of Medication Regimen: The subject was receiving antipsychotic therapy with one or two atypical antipsychotic medications for at least 8 weeks prior to Day −1 Visit. In addition, the subject had no symptom-related changes in antipsychotic or antidepressant medications from 8 weeks prior to Day −1 and no changes in dose(s) of those medications for any reason from 4 weeks prior to Day −1.
   Severity of Symptoms: Core positive symptoms were no worse than moderate in severity, extrapyramidal symptoms (EPS) were no worse than mild in severity, and depressive symptoms are not consistent with a major depressive episode from the start of Screening through the end of the Prospective Stabilization Period, as defined by the following:
      Positive and Negative Syndrome Scale (PANSS) item scores of ≦4 each for delusions (P1), conceptual disorganization (P2), hallucinatory behavior (P3), and excitement (P4);
      In the Investigator's judgment, no clinically significant EPS at the Initial Screening Visit, a Day −1 Severity of Abnormal Movements item score of ≦2 on the Abnormal Involuntary Movement Scale (AIMS), and a Day −1 Global Clinical Rating of Akathisia item score of 2 on the Barnes Akathisia. Rating Scale (BAS);
      Calgary Depression Scale for Schizophrenia (CDSS) total score of ≦10 at Screening.
5. Had been diagnosed with or treated for schizophrenia for at least 2 years prior to Screening.
6. If female, must be either not of childbearing potential, defined as postmenopausal for at least 2 years or surgically sterile (bilateral tubal ligation, bilateral oophorectomy, or hysterectomy), or of childbearing potential and agree to using a double barrier method (physical barrier, e.g., condom or IUD, and chemical barrier, e.g., birth control pills, jellies or foams) from the time of the Initial Screening Visit through the end of the Follow-up Period. Diaphragm must be used with spermicidal foam or jelly. The combination of diaphragm and spermicidal substance counts as a single barrier.
7. If a female is of childbearing potential, the result of a serum pregnancy test performed at the initial Screening Visit is negative, and the subject does not plan to become pregnant during the study.
8. If female, is not breast-feeding.
9. If male, is surgically sterile (vasectomy), is sexually inactive, or agrees to using a barrier method (condom) of birth control from the time of the Initial Screening Visit through the end of the Follow-up Period.
10. Has had continuity in psychiatric care (mental health system, clinic or physician), as indicated by available medical records or a corroborating clinician or case worker for at least 6 months prior to Screening.

### Randomization, Medication Dosing, and Dispensing

Subjects were randomized in a 1:1:1 ratio with placebo, 10 mg QD Compound A, of 25 mg Compound A. Each subject was instructed to take study drug once-daily in the morning for 12 weeks. Each daily dose was preferably taken with food. The subject and investigator were blinded to the treatment assignment throughout the study. The treatment assignments for the study subjects are shown below in Table 1.

TABLE 1
Treatment Assignments

| Treatment Group | N | Dose |
|---|---|---|
| A | 70 | Placebo QD |
| B | 70 | 10 mg Compound A QD |
| C | 70 | 25 mg Compound A QD |

### Visits and Measurements

Subjects completed 2 visits during the screening period. Study site personnel contacted each subject by telephone on Day 21, 35, and 70 of the 84-day treatment period to discuss study drug compliance, antipsychotic medication compliance, concomitant medication use, substance use, and any adverse events.

### Endpoints and Measures of Outcome

The primary endpoint was the MCCB composite score, and the primary endpoint analysis was the change on the MCCB composite score from baseline to endpoint versus placebo. Other secondary measures included the MCCB domains, the NSA-16, the CANTAB cognition battery (measured at different time points from the MCCB), and the UPSA-2. The Positive and Negative Syndrome Scale (PANSS) was included to document stability in schizophrenia symptomatology.

### MATRICS Consensus Cognitive Battery (MCCB)

The MCCB was developed by a consortium of academic, industry, the Food and Drug Administration (FDA) and National Institute of Mental Health (NUMB) members called Measurement and Treatment Research to Improve Cognition in Schizophrenia (MATRICS). The battery was established in a multiple phase process that involved experts identifying cognitive tests in the literature that had shown deficits in schizophrenia, use of factor analysis to identify key domains of cognitive deficits in schizophrenia, and then empirically identifying the best tests for each domain based on reliability, validity and feasibility for use in clinical trials. The FDA has endorsed the MCCB as an appropriate outcome measure for Phase 3 CDS trials.

The MCCB comprises 10 tests (Trail Making Test Part A, Brief Assessment of Cognition in Schizophrenia Synibol Coding, Hopkins Verbal Learning Test—Revised Immediate Recall Three Trial Learning, Wechsler Memory Scale 3^{rd} Ed. Spatial Span, Letter-Number Span, Neuropsychological Assessment Battery Mazes, Brief Visuospatial Memory Test—Revised, Category Fluency Test Animal Naming, Mayer-Solvay-Caruso Emotional Intelligence Test Managing Emotions, Continuous Performance Test Identical Pairs) of cognitive functioning and assesses seven domains of cognition (speed of processing, verbal learning, working memory, reasoning and problem solving, visual learning, attention/vigilance and social cognition). Repeated administration of the MCCB tests of verbal learning, visual learning and reasoning may result in large content-related practice effects. Therefore, alternate versions of these tests were used in order to minimize practice effects. In order to control for alternate form difficulty, the sequence of the alternate forms were counterbalanced across patients so that at study end, each form was given at each visit a similar number of times. Each site received a schedule for alternate forms from NeuroCog Trials. The MCCB showed good test-retest reliability and discriminated patients with schizophrenia from normal subjects and correlates with functional status. The MCCB took approximately 60 to 90 minutes to administer and was given at the times indicated in on Days 14, 28, 56, 84 and 98.

### UCSD Performance-Based Skills Assessment-2 (UPSA-2)

The UCSD Performance-Based Skills Assesstnent-2 (UPSA-2) is a role-play test designed for subjects with schizophrenia to evaluate cognitive functional capacity in six selected domains of basic living skills. These areas include Organization/Planning Financial Skills, Communication, Transportation, Household Management, and Medication Management. Patients being tested utilize props to demonstrate how they perform everyday activities and are assessed on their actual performance. Scores were obtained for each subtest, and the total score was the sum of these subtests. The UPSA-2 demonstrated established reliability and validity and significantly correlated with the MCCB. The UPSA-2 required an average of 30 minutes to administer. The UPSA-2 was administered in on Days 14, 28, 56, 84 and 98.

### Cambridge Neuropsychological Test Automated Battery (CANTAB) for Schizophrenia

The Cambridge Neuropsychological Test Automated Battery (CANTAB) is a computer-based cognitive assessment system consisting of a battery of neuropsychological tests, administered to subjects using a touch screen computer. The CANTAB battery shows good test/retest reliability and discriminates patients with schizophrenia from normal subjects. The battery also shows pharmacologic sensitivity to a number of compounds including atomoxetine. The CANTAB computerized system will be employed to explore the effects of Compound A on cognition. The tests assess the following cognitive domains: executive function, spatial memory, attention and episodic memory. The CANTAB battery took approximately 40 minutes to administer and was given on Days 14, 28, 56, 84 and 98.

The cognitive tests included in this version of the CANTAB battery are as follows:
Motor Screening
Rapid Visual Information Processing
5 Choice Serial Reaction Time
Spatial Working memory
Paired Associates Learning
Stockings of Cambridge
Emotion Recognition Task
Delayed Match to Sample

### Statistical Analysis

Individual Compound A plasma concentrations at each study visit were tabulated and summarized with appropriate statistical methods. Population pharmacokinetic analyses were performed using the actual sampling time relative to dosing. Pharmacokinetic models were built using a non-linear mixed-effect modeling (NONMEM) approach with the NONMEM software (Version VI, or higher version). The structure of the starting pharmacokinetic model will be based on the pharmacokinetic analysis of data from previous studies. Apparent oral clearance (CL/F) and apparent volume of distribution (Vₛₛ/F) of Compound A were the pharmacokinetic parameters of major interest in the NONMEM analyses. If necessary, other parameters, including the parameters describing absorption characteristics, were fixed in the analysis,

### Results

Subject Characteristics and Disposition

A total of 207 subjects were randomized. Four subjects did not receive a study drug following randomizations and were not included in the efficacy analyses. The disposition of subjects is shown in Table 2.

TABLE 2
Preliminary Disposition of Subjects

| | Treatment Group, n (%) | | | | |
|---|---|---|---|---|---|
| | | Compound A | | | |
| Subjects: | Placebo | 10 mg QD | 25 mg QD | Overall | Total |

| | | | | | | | | | | |
|---|---|---|---|---|---|---|---|---|---|---|
| Randomized | 68 | | 70 | | 69 | | 139 | | 207 | |
| Treated | 67 | | 69 | | 67 | | 136 | | 203 | |
| Completed study | 56 | (83.6) | 60 | (87.0) | 49 | (73.1) | 109 | (80.1) | 165 | (81.3) |
| Primary reason for | 11 | (16.4) | 9 | (13.0) | 18 | (26.9) | 27 | (19.9) | 38 | (18.7) |
| discontinuation | | | | | | | | | | |
| Adverse event | 5 | (7.5) | 4 | (5.8) | 3 | (4.5) | 7 | (5.1) | 12 | (5.9) |
| Withdrew consent | 2 | (3.0) | 1 | (1.4) | 3 | (4.5) | 4 | (2.9) | 6 | (3.0) |
| Lost to follow-up | 1 | (1.5) | 1 | (1.4) | 4 | (6.0) | 5 | (3.7) | 6 | (3.0) |
| Other | 3 | (4.5) | 3 | (4.3) | 8 | (11.9) | 11 | (8.1) | 14 | (6.9) |

Note:
Percentages are based on the number of treated subjects.

One hundred sixty-five (1.3%) of the treated subjects completed the study. Of the subjects who prematurely terminated, 12 (5.9%) were primarily discontinued due to adverse events and 14 (6.9%) were primarily discontinued for other reasons. The Compound A 25 mg group had the lowest completion rate, with 73.1%. The imbalance was largely in the “other” category; the “other” reasons in this treatment group were heterogeneous, and no clear pattern could be ascribed to the discontinuations in the category. Baseline characteristics of the patients are shown in Table 3.

TABLE 3

| | Overall |
|---|---|
| Characteristic | N = 203 |
| Age (years), mean | 42.3 |
| Gender, male, n (%) | 64.5% |
| Average age onset of symptoms | 22.3 years |
| Average age at diagnosis | 25.7 years |
| MATRICS Cognition Consensus Battery Score | 27.4 |
| (mean) | |
| University of California Performance-based Skills | 86.3 |
| Assessment Score (Mean) | |
| Positive and Negative Syndrome Scale Score | 64.2 |
| (mean) | |

### Efficacy and Safety

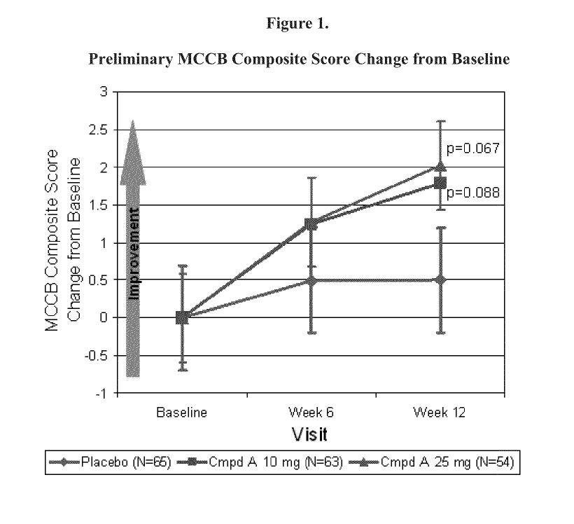

The mean baseline MCCB composite score in this study was 27.4 (SD 12.77) (the scoring has been standardized such that the mean [SD] value in a healthy population is 50 [10]). In the intent-to-treat (ITT) analysis, the change from baseline to Week 12 in MCCB composite score for the Compound A 10 mg and 25 mg dose groups (LS mean+1.79 and +2.02, respectively) trended towards improvement (P=0.088 and P=0.067, respectively) versus placebo (LS mean+0.50) as shown in FIG. 1. The results on the composite score were driven by 3 domains: verbal learning (P=0.063 in 25 mg Lump); working memory (P=0.054 in 25 mg group) and attention (P=0.036 in 25 mg group). In addition, dose response relationships were observed for 6 of the 7 domains; with the lone exception being reasoning. At Week 6, the LS mean difference versus placebo was greater for in both Compound A dose groups for 5 of the 7 MCCB domains (Table 4). The results were consistent on the CANTAB battery.

TABLE 4
Repeated-Measure Analysis of Change from Baseline to Week 6
and Week 12 for MCCB Domain Scores (ITT Data Set)

| | | | LS Mean (SE) | Difference from Placebo | | | |
|---|---|---|---|---|---|---|---|
| MCCB Domain | | | of Change | LS Mean | | | |
| Visit | | Observed | from | (SE) | | | |
| Treatment | N | Mean (SD) | Baseline | of Difference | 90% CI | P value^{a} | |
| Speed of Processing | | | | | | | |
| Baseline | | | | | | | |
| Placebo | 65 | 29.63 (12.05) | | | | | |
| Compound A | 63 | 34.89 (13.86) | | | | | |
| 10 mg | | | | | | | |
| Compound A | 54 | 34.56 (10.97) | | | | | |
| 25 mg | | | | | | | |
| Change to Week 6 | | | | | | | |
| Placebo | 65 | 1.22 (7.02) | 0.40 (0.74) | | | | |
| Compound A | 63 | 1.00 (5.49) | 1.13 (0.74) | 0.74 (1.04) | (−0.98, 2.45) | 0.239 | |
| 10 mg | | | | | | | |
| Compound A | 54 | 2.56 (5.36) | 2.24 (0.80) | 1.84 (1.08) | (0.06, 3.63) | 0.045 | * |
| 25 mg | | | | | | | |
| Change to Week 12 | | | | | | | |
| Placebo | 56 | 1.66 (6.91) | 1.28 (0.87) | | | | |
| Compound A | 60 | 2.33 (6.05) | 2.36 (0.85) | 1.08 (1.20) | (−0.91, 3.07) | 0.185 | |
| 10 mg | | | | | | | |
| Compound A | 49 | 1.73 (7.28) | 1.66 (0.93) | 0.37 (1.26) | (−1.72, 2.47) | 0.384 | |
| 25 mg | | | | | | | |
| Verbal Learning | | | | | | | |
| Baseline | | | | | | | |
| Placebo | 65 | 35.14 (7.63) | | | | | |
| Compound A | 63 | 35.35 (7.57) | | | | | |
| 10 mg | | | | | | | |
| Compound A | 54 | 37.35 (9.55) | | | | | |
| 25 mg | | | | | | | |
| Change to Week 6 | | | | | | | |
| Placebo | 65 | 0.82 (5.91) | 0.07 (0.80) | | | | |
| Compound A | 63 | 0.68 (7.99) | 0.31 (0.82) | 0.24 (1.12) | (−1.62, 2.10) | 0.416 | |
| 10 mg | | | | | | | |
| Compound A | 54 | 0.41 (9.24) | 0.56 (0.88) | 0.49 (1.18) | (−1.47, 2.45) | 0.339 | |
| 25 mg | | | | | | | |
| Change to Week 12 | | | | | | | |
| Placebo | 56 | 1.18 (6.80) | 0.47 (0.80) | | | | |
| Compound A | 60 | 1.28 (7.06) | 1.08 (0.79) | 0.61 (1.10) | (−1.21, 2.43) | 0.290 | |
| 10 mg | | | | | | | |
| Compound A | 49 | 2.24 (8.03) | 2.27 (0.87) | 1.80 (1.17) | (−0.14, 3.74) | 0.063 | + |
| 25 mg | | | | | | | |
| Working Memory | | | | | | | |
| Baseline | | | | | | | |
| Placebo | 65 | 32.52 (11.79) | | | | | |
| Compound A | 63 | 34.43 (11.11) | | | | | |
| 10 mg | | | | | | | |
| Compound A | 54 | 36.09 (10.55) | | | | | |
| 25 mg | | | | | | | |
| Change to Week 6 | | | | | | | |
| Placebo | 65 | −0.23 (5.52) | −0.51 (0.74) | | | | |
| Compound A | 63 | 0.27 (5.15) | 0.35 (0.75) | 0.86 (1.03) | (−0.84, 2.56) | 0.201 | |
| 10 mg | | | | | | | |
| Compound A | 54 | 1.69 (7.43) | 1.88 (0.81) | 2.39 (1.08) | (0.60, 4.18) | 0.014 | * |
| 25 mg | | | | | | | |
| Change to Week 12 | | | | | | | |
| Placebo | 56 | 0.29 (5.76) | 0.11 (0.86) | | | | |
| Compound A | 60 | 0.83 (7.04) | 0.92 (0.84) | 0.80 (1.18) | (−1.15, 2.76) | 0.249 | |
| 10 mg | | | | | | | |
| Compound A | 49 | 1.98 (6.84) | 2.14 (0.93) | 2.03 (1.25) | (−0.04, 4.11) | 0.054 | + |
| 25 mg | | | | | | | |
| Reasoning and Problem Solving | | | | | | | |
| Baseline | | | | | | | |
| Placebo | 65 | 39.17 (9.43) | | | | | |
| Compound A | 63 | 41.16 (9.78) | | | | | |
| 10 mg | | | | | | | |
| Compound A | 54 | 40.89 (8.87) | | | | | |
| 25 mg | | | | | | | |
| Change to Week 6 | | | | | | | |
| Placebo | 65 | 0.00 (5.56) | −0.04 (0.74) | | | | |
| Compound A | 63 | 0.08 (5.57) | 0.49 (0.75) | 0.53 (1.03) | (−1.18, 2.23) | 0.305 | |
| 10 mg | | | | | | | |
| Compound A | 54 | 2.11 (7.06) | 2.18 (0.81) | 2.22 (1.08) | (0.44, 4.01) | 0.021 | * |
| 25 mg | | | | | | | |
| Change to Week 2 | | | | | | | |
| Placebo | 56 | 0.61 (7.78) | 0.75 (0.85) | | | | |
| Compound A | 60 | 1.70 (6.03) | 1.99 (0.84) | 1.24 (1.18) | (−0.71, 3.19) | 0.147 | |
| 10 mg | | | | | | | |
| Compound A | 49 | 2.00 (6.25) | 1.95 (0.92) | 1.20 (1.24) | (−0.86, 3.25) | 0.169 | |
| 25 mg | | | | | | | |
| Visual Learning | | | | | | | |
| Baseline | | | | | | | |
| Placebo | 65 | 34.32 (12.65) | | | | | |
| Compound A | 63 | 38.30 (12.00) | | | | | |
| 10 mg | | | | | | | |
| Compound A | 54 | 39.37 (12.90) | | | | | |
| 25 mg | | | | | | | |
| Change to Week 6 | | | | | | | |
| Placebo | 65 | 0.75 (9.41) | −0.15 (1.12) | | | | |
| Compound A | 63 | 1.35 (9.36) | 1.59 (1.13) | 1.74 (1.57) | (−0.85, 4.34) | 0.134 | |
| 10 mg | | | | | | | |
| Compound A | 54 | −1.06 (9.04) | −1.15 (1.22) | −1.00 (1.65) | (−3.73, 1.72) | 0.729 | |
| 25 mg | | | | | | | |
| Change to Week 12 | | | | | | | |
| Placebo | 56 | 0.46 (10.89) | −0.44 (1.15) | | | | |
| Compound A | 60 | 0.05 (8.06) | 0.29 (1.12) | 0.73 (1.59) | (−1.90, 3.36) | 0.324 | |
| 10 mg | | | | | | | |
| Compound A | 49 | 1.24 (8.49) | 0.83 (1.23) | 1.27 (1.68) | (−1.51, 4.05) | 0.226 | |
| 25 mg | | | | | | | |
| Attention/Vigilance | | | | | | | |
| Baseline | | | | | | | |
| Placebo | 65 | 35.28 (11.67) | | | | | |
| Compound A | 63 | 36.30 (13.35) | | | | | |
| 10 mg | | | | | | | |
| Compound A | 54 | 37.02 (11.80) | | | | | |
| 25 mg | | | | | | | |
| Change to Week 6 | | | | | | | |
| Placebo | 65 | −0.25 (7.26) | −0.80 (0.96) | | | | |
| Compound A | 63 | 0.83 (7.62) | 0.49 (0.98) | 1.29 (1.35) | (−0.93, 3.52) | 0.169 | |
| 10 mg | | | | | | | |
| Compound A | 54 | 1.09 (9.04) | 0.91 (1.06) | 1.71 (1.41) | (−0.63, 4.05) | 0.114 | |
| 25 mg | | | | | | | |
| Change to Week 12 | | | | | | | |
| Placebo | 56 | −1.52 (7.52) | −1.99 (1.08) | | | | |
| Compound A | 60 | 1.15 (8.46) | 0.64 (1.06) | 2.63 (1.49) | (0.17, 5.09) | 0.040 | * |
| 10 mg | | | | | | | |
| Compound A | 49 | 0.88 (9.09) | 0.86 (1.16) | 2.85 (1.57) | (0.25, 5.45) | 0.036 | * |
| 25 mg | | | | | | | |
| Social Cognition | | | | | | | |
| Baseline | | | | | | | |
| Placebo | 65 | 35.98 (13.48) | | | | | |
| Compound A | 63 | 35.83 (12.82) | | | | | |
| 10 mg | | | | | | | |
| Compound A | 54 | 38.02 (13.81) | | | | | |
| 25 mg | | | | | | | |
| Change to Week 6 | | | | | | | |
| Placebo | 65 | 1.48 (6.95) | 1.50 (0.95) | | | | |
| Compound A | 63 | 1.14 (7.87) | 1.04 (0.97) | −0.46 (1.34) | (−2.67, 1.75) | 0.636 | |
| 10 mg | | | | | | | |
| Compound A | 54 | 0.06 (8.00) | 0.30 (1.05) | −1.21 (1.40) | (−3.53, 1.11) | 0.805 | |
| 25 mg | | | | | | | |
| Change to Week 12 | | | | | | | |
| Placebo | 56 | 0.43 (7.87) | 0.59 (1.10) | | | | |
| Compound A | 60 | 1.38 (6.51) | 0.99 (1.08) | 0.40 (1.52) | (−2.11, 2.92) | 0.395 | |
| 10 mg | | | | | | | |
| Compound A | 49 | −0.14 (10.81) | 0.19 (1.19) | −0.40 (1.61) | (−3.06, 2.27) | 0.597 | |
| 25 mg | | | | | | | |

MCCB = MATRICS Consensus Cognitive Battery,
SD = standard deviation,
LS = least squares,
SE = standard error,
CI = confidence interval
^{a}One-sided P value from repeated measures model with treatment, site, visit, baseline score, interactions of treatment and visit, and interaction of baseline score and visit; covariance structure is unstructured.
Note:
The MCCB domain scores are age- and gender-adjusted T-scores with a population mean of 50 and standard deviation of 10. An increasing MCCB domain score represents improvement from baseline.

Early improvement in cognition with Compound A is suggested by an increase from baseline to Week 6 in MCCB composite score for both the 10 mg group (LS mean+1.25) and the 25 mg dose group (LS mean+1.27) relative to placebo (LS mean+0.49) according to MNIRIVI analysis, although the difference between each Compound A dose group and placebo did not reach the level of statistical significance.

No significance was found for either dose group on the UPSA-2 composite score; however, a statistically significant improvement or statistical trend for improvement was seen on the medication management (P=0.094 at 10 mg), comprehension/planning (P=0.102 at 10 mg and P=0.005 at 25 mg), and household skills (P=0.096 at 10 mg) subscale scores (Table 5).

TABLE 5
Analysis of Covariance of Change from Baseline to Final Evaluation for
UPSA-2 Total Score

| | | | | | Difference from Placebo | | |
|---|---|---|---|---|---|---|---|
| Treatment | | Observed Mean (SD) | | LS Mean (SE) | LS Mean (SE) | | |
| Group | N | Baseline | Final | of Change | of Difference | 90% CI | P value^{a} |
| Placebo | 60 | 80.18 (18.70) | 82.60 (19.57) | 2.27 (1.57) | | | |
| Compound A | 62 | 85.94 (13.68) | 87.98 (20.36) | 2.78 (1.52) | 0.51 (2.15) | (−3.04, 4.07) | 0.406 |
| 10 mg | | | | | | | |
| Compound A | 52 | 92.17 (13.16) | 94.29 (15.85) | 2.89 (1.70) | 0.63 (2.34) | (−3.24, 4.49) | 0.394 |
| 25 mg | | | | | | | |

### Example 2

### Experimental Details

### Clinical Study B: A Randomized, Double-Blind, Placebo-Controlled Dose-Ranging, Parallel-Group, Phase 2 Study of the Efficacy and Safety of Compound A in Treatment of Cognitive Deficits of Schizophrenia (CDS)

This is a multicenter, randomized, double-blind, placebo-controlled, dose-ranging, parallel-group, study designed to evaluate the efficacy and safety of Compound A in the treatment of cognitive deficits in schizophrenia. (CDS) in nonsmokers. Approximately 430 subjects will be randomized to one of four treatment groups (Compound A 25 mg, Compound A 50 mg, Compound A 75 mg, or placebo) for a 24 week double-blind treatment period. Patients will be administered capsules orally.

Inclusion Criteria for the study subjects include:

1. Male or female between 20 and 55 years of age, inclusive, at 1 time of randomization.

2. Has a current DSM-IV-TR diagnosis of schizophrenia confirmed by the M.I.N.I.

3. Is receiving one or two antipsychotic medications, restricted to any of the following allowable agents: amisulpride, aripiprazole, asenapine, lurasidone, olanzapine, paliperidone, quetiapine, risperidone, ziprasidone, haloperidol, fluphenazine and perphenazine.

4. Is clinically stable in the residual phase of the illness, as defined by the following criteria:
Level of Care: The subject has had no psychiatric inpatient hospitalization, no overnight crisis stabilization, no emergency room visit for psychiatric symptoms, and no other overt signs of destabilization in the 4 months prior to Screening Visit 1.
Stability of Medication Regimen: The subject has had no symptom-related changes in antipsychotic, antidepressant, or mood-stabilizing medications within 8 weeks prior to Day −1 and no changes in dose(s) of those medications for any reason within 4 weeks prior to Day −1.
Severity of Symptoms: Core positive symptoms are no worse than moderate in severity, extrapyramidal symptoms (EPS) are no worse than mild in severity, and depressive symptoms are not consistent with a major depressive episode from the start of Screening through the end of the Prospective Stabilization Period, as defined by the following: Positive and Negative Syndrome Scale (PANSS) item scores of ≦5 each for delusions (P1), conceptual disorganization (P2), hallucinatory behavior (P3), and excitement (P4); In the Investigator's judgment, no clinically significant EPS at Screening Visit 1, a Severity of Abnormal Movements item score of ≦2 on the Abnormal Involuntary Movement Scale (AIMS) at Day −1, and a Global Clinical Rating of Akathisia score of ≦2 on the Barnes Akathisia Rating Scale (BA S) at Day −1; Calgary Depression Scale for Schizophrenia (CDSS) total score of ≦10 at Screening Visit 1.

5. Has been diagnosed with or treated for schizophrenia for at east 2 years prior to Screening Visit 1.

Exclusion Criteria, for the study subjects include:

1. In the Investigator's judgment, has a current or past diagnosis of schizoaffective disorder, bipolar disorder, manic episode, dementia, post traumatic stress disorder, or obsessive-compulsive disorder, or the subject has a current major depressive episode.

2. Has a positive urine drug screen for cocaine, phencyclidine (PCP), opiates (unless duly prescribed), benzodiazepines (unless duly prescribed), marijuana, or amphetamines at Screening Visit 1, Screening Visit 2 or Day −1.

3. Has a body mass index (BMI)>40 kg/m2 at Screening Visit 1. BMI is calculated as weight in kilograms divided by the square of height in meters (kg/m2).

4. Has a current or past history of seizures, with the exception of a single febrile seizure occurring prior to 6 years of age.

5. Has a clinically significant abnormal ECG at Screening Visit 1 as determined by the Investigator.

6. Has any risk factors for Torsades de Pointes (TdP)

Based on the data available for Compound A, it is anticipated that doses of 50 mg QD and 75 mg QD will demonstrate efficacy in the tested subjects as effectively or more effectively than a 25 mg QD dose of Compound A.

in summary, Compound A has demonstrated a signal for efficacy in the symptomatic treatment of AD in the Phase 2a study and appears to be well tolerated in subjects with schizophrenia in doses up to 25 mg QD, including 10 mg QD and 25 mg QD, and can be anticipated to demonstrate efficacy in improving cognitive deficits of schizophrenia at doses of 50 mg QD and 75 mg QD.

It is understood that the foregoing detailed description and accompanying examples are merely illustrative and are not to be taken as limitations upon the scope of the invention, Which is defined solely by the appended claims and their equivalents.

What is claimed is:

1. Use of a selective agonist of neuronal nicotinic acetylcholine receptor α7 subtype for the preparation of a medicament for improving symptoms of cognitive deficit associated schizophrenia in a patient.

2. The use of claim 1, wherein e selective agonist of neuronal nicotinic acetylcholine receptor α7 subtype is (4s)-4-(5-phenyl-1,3,4-thiadiazol-2-yloxy)-1-azatricyclo[3.3.1.1^{3,7}]decane, N-[2-(pyridin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide or a salt thereof.

3. The use of claim 1, wherein the selective agonist of neuronal nicotinic acetylcholine receptor α7 subtype is administered in an amount of from about 6 mg to about 150 mg to a patient.

4. The use of claim 1, wherein the selective agonist of neuronal nicotinic acetylcholine receptor α7 subtype is administered in an amount of 10, 25, 50, or 75 mg to a patient once a day.

5. A method for improving cognitive symptoms associated with schizophrenia or a related schizophrenia spectrum psychotic disorders, comprising the step of administering a therapeutically effective amount of a selective agonist of neuronal nicotinic acetylcholine receptor α7 subtype, or a pharmaceutically acceptable salt thereof, to a patient in need of such treatment.

6. The method of claim 5, wherein the selective agonist of neuronal nicotinic acetylcholine receptor α7 subtype is (4s)-4-(5-phenyl-1,3,4-thiadiazol-2-yloxy)-1-azatricyclo[3.3.1.1^{3,7}]decane, N-[2-(pyridin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide or a salt thereof.

7. The method of claim 5, wherein the selective agonist of neuronal nicotinic acetylcholine receptor α7 subtype is administered in an amount of from about 10 mg to about 75 mg to a patient.

8. The method of claim 7, wherein the selective agonist of neuronal nicotinic acetylcholine receptor α7 subtype is administered in an amount of 10, 25, 50, or 75 mg to a patient once daily.

9. A pharmaceutical composition for use in the treatment of cognitive symptoms of schizophrenia comprising administering a therapeutically effective amount of a selective agonist of neuronal nicotinic acetylcholine receptor α7 subtype and a pharmaceutically acceptable excipient.

10. The composition of claim 1 further comprising a pharmaceutically acceptable hydrophilic polymer and a pharmaceutically acceptable surfactant.

11. The composition of claim 10, wherein the selective agonist of neuronal nicotinic acetylcholine receptor α7 subtype is (4s)-4-(5-phenyl-1,3,4-thiadiazol-2-yloxy)-1-azatricyclo[3.3.1.1^{3,7}]decane, N-[2-(pyridin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide or a salt thereof.

12. The composition of claim 11, wherein the selective agonist of neuronal nicotinic acetylcholine receptor α7 subtype is in an amount of from about 25 mg to about 75 mg.

13. The composition of claim 11, wherein the selective agonist of neuronal nicotinic acetylcholine receptor α7 subtype is in an amount of from about 25, 50, or 75 mg.
